(19) United States
(12) Patent Application Publication
(10) Pub. No.: US 2007/0196816 A1
(43) Pub. Date: Aug. 23, 2007

(54) Engineered stimulus-responsive switches

(76) Inventors: John Jacob Schwartz, Newtonville, MA (US); Joseph Jacobson, Newton, MA (US); Ruchira Das Gupta, Jamaica Plain, MA (US)

Correspondence Address: GOODWIN PROCTER LLP;PATENT ADMINISTRATOR, EXCHANGE PLACE, BOSTON, MA, 02109-2881, US

(21) Appl. No.: 11/705,565

(22) Filed: Feb. 12, 2007

Related U.S. Application Data
(62) Division of application No. 10/032,827, filed on Oct. 23, 2001, now abandoned.
(60) Provisional application No. 60/242,546, filed on Oct. 23, 2000.

(51) Int. Cl. C12Q 1/00; G06F 19/00; C07H 21/04; C07K 14/435; G01N 33/48; C12N 15/74

(52) U.S. Cl. 435/4; 435/254.2; 435/483; 530/324; 536/23.2; 702/19

(57) ABSTRACT

Ligand-responsive chimeric proteins are engineered to cause a detectable output in response to a preselected stimulus. The engineered chimeric proteins are useful in industrial, commercial, medical, and scientific fields as a tool for programming a cellular response to a stimulus of choice and for use with in vitro assays. The engineered chimeric proteins include a detection domain and an interaction domain. Interaction of the engineered chimeric protein with a target biomolecule is modulated by the presence or absence of the preselected stimulus.

### REFERENCE TO RELATED APPLICATIONS

This application claims priority to U.S. Ser. No. 60/242,546, filed Oct. 23, 2000, the complete disclosure of which is herein incorporated by reference.

### BACKGROUND OF THE INVENTION

A living cell is an awe-inspiring machine. Every microscopic cell contains within itself the information required to reproduce itself, grow, nourish itself, adapt to its environment, and, often, to alter its environment and/or to move to a new location. The cell carries this information in its genetic code and regulates its activities, among other ways, by controlling which genes are transcribed at any one time. A bacterium, for example, may be able to nourish itself by consuming any one of a number of sugars (e.g. lactose or glucose), but may only transcribe genes that help it to consume lactose when the cell finds lactose to consume. A gene includes at least two elements: a “coding region” containing the information to be transcribed as an RNA molecule is synthesized, and one or more control elements that regulate synthesis of RNA. A control element, often referred to as a “promoter element,” “operator element,” or “enhancer element,” may be located within the coding region, although at least one control element is normally found outside the coding region. The control elements make it easier or harder for RNA polymerase to find the gene and to begin transcription. RNA polymerase generally needs a number of positive control elements to help it to find the beginning of a gene. RNA polymerase may directly interact with the DNA sequence of a positive control element. Often, however, another protein (referred to generally as a “transcription factor”; a transcription factor that promotes transcription is also called an “activator”) may act as an intermediary, binding to the DNA sequence of the positive control element and to the RNA polymerase. The transcription factor stabilizes RNA polymerase at the beginning of the gene, thereby to facilitate transcription. A negative control element may also interact with a transcription factor (in this instance often called a “repressor”) and functions to hinder transcription, for example, by physically blocking RNA polymerase from associating with or transcribing the gene (“steric hindrance”), by modifying the structure of the DNA to make it less accessible to the RNA polymerase, by interfering with the action of an activator, or by modifying the RNA polymerase itself.

One common way in which cells regulate transcription of a gene is by modifying the presence or availability of active repressors and activators. For example, in mammalian cells, the RB repressor controls the transcription of a number of genes required for DNA synthesis. Before DNA synthesis, the RB repressor is phosphorylated, which inactivates the repressor, and transcription of the DNA synthesis genes begins. In E. coli bacteria, the lac repressor inhibits transcription of the β-galactosidase enzyme, which is used in consuming lactose. Lactose, if present, binds and inactivates the lac repressor, permitting synthesis of β-galactosidase and consumption of the lactose. Often, the availability of a transcription factor is modified by its own transcription. For example, a number of mammalian developmental pathways that create and maintain tissue organization (e.g. proper placement and form of arms, legs, organs, etc.) involve cascades of transcription factors affecting each other's (and their own) transcription.

The ability of a cell to sense its surroundings and to respond by executing a complex program of responses is an amazingly sophisticated and powerful tool. If a cell could be engineered to carry a different program of responses, a program designed de novo to carry out a useful process in response to a stimulus of choice, such a tool would be of enormous value in medical diagnosis and treatment, chemical synthesis, environmental remediation, pharmaceutical screening and synthesis, medical research, and nanomanufacturing, among other fields.

### SUMMARY OF THE INVENTION

It has now been discovered that the accumulated knowledge of the structure of biomolecules and of the mechanisms of regulation of transcription and translation permits the engineering of a novel class of engineered chimeric proteins that can detect and respond to a preselected stimulus. These engineered chimeric proteins are tools that can, for example, be used to reprogram the transcriptional machinery of a cell or of an acellular system to respond to any desired signal input(s). The engineered chimeric proteins may behave as classical transcription factors and/or may regulate the activity of classical and/or artificial transcription factors. Because the engineered chimeric proteins can be engineered to respond to an arbitrary and preselected biophysical stimulus (e.g. a ligand), a cell engineered to contain the engineered chimeric protein can alter its transcriptional program in response to such a stimulus. Furthermore, different engineered chimeric proteins can be combined in the same cell, or in a collection of cells, permitting the creation of an entire transcriptional program designed to provide whatever outputs are desired in response to the selected input signals. Alternatively, cell-free in vitro systems making use of these proteins may be envisioned. These systems would not be under the same rigorous biological constraints associated with cell-based systems (e.g. temperature, pH, osmolality, etc.)

The enormous flexibility of this approach allows a cell to execute a program in ways not unlike the execution of a computer program by a microprocessor. This permits the intelligent design of systems that have never before existed in molecular biology, such as, for example, mechanisms for counting the number of times a cell is exposed to a carcinogen and to emit light after the third exposure, or mechanisms for depositing a conductive material on a substrate in a particular pattern, or mechanisms for releasing a pharmaceutical agent into the bloodstream three times daily. As in computer programming, the possibilities are limited primarily by the ingenuity of the programmer. Unlike a computer, however, the cell is its own factory; the output of the cell need not be a mere digital signal (although it could be), but can include synthesis and release of an end product. The cell can also be engineered to include a self-destruct signal. Thus, a bacterium for use in waste management could be engineered to consume a polymer, but could include a transcriptional switch to kill the bacterium in response to a preselected ligand if the bacterium escaped into the environment. Similarly, a cell could be engineered to cleanse the blood vessels of atherosclerotic plaques by applying enzymes that attack the plaques, and to die when its work was complete or in response to a chemical injected into the bloodstream.

We have discovered that principles of modular design can be applied to biological and biochemical systems to engineer stimulus-responsive proteins whose interaction with a target biomolecule (such as a DNA, an RNA, a protein, a carbohydrate, or other biomolecule) is regulated by the presence or absence of a preselected stimulus. Thus, the engineered chimeric protein (and engineered systems using the engineered chimeric protein) senses and then acts to transform its environment. These modular design principles, which can be used to leverage molecular biology, structural biology, modeling technologies and molecular genetics, significantly reduce the time and expense traditionally associated with biological design, facilitating the engineering of a wider range of tools with greater precision, sensitivity, and versatility.

In one embodiment, an engineered chimeric protein of the invention includes at least two domains: an interaction domain capable of binding a target biomolecule and a detection domain that includes a peptide that recognizes and is responsive to a stimulus. The stimulus may be, for example, a change in a concentration of a ligand that binds the peptide, a change in a thermodynamic state (e.g. temperature, pressure, etc.) that alters the conformation of the peptide, a change in electromagnetic radiation (e.g. a pulse of visible light or of radio waves) detected by the peptide, or other stimulus (e.g. a change in an oxidation state). The peptide is no more than one hundred amino acids long, and is preferably smaller (e.g. no more than eighty, no more than sixty, no more than forty, or no more than twenty amino acids long) to minimize any risk that the peptide will unduly disrupt the structure of the interaction domain. The peptide includes an amino acid sequence selected so the stimulus causes a change (e.g. a steric or allosteric change, a change in charge or oxidation state, etc.) in the engineered chimeric protein, and that change regulates binding of the interaction domain to the target biomolecule. The peptide also is bonded at a position in the interaction domain selected to permit that change in response to the stimulus.

In another embodiment, the detection domain is a ligand binding domain including a peptide that binds to a ligand, and an interaction domain capable of binding a target biomolecule. Selection of the peptide is informed by a recombinant display technique. “Recombinant display technique,” as used herein, refers to any method for selecting or screening a library for peptides with an affinity for a ligand, including, for example, phage display, single chain antibody display, retroviral display, bacterial surface display, yeast surface display, ribosome display, two-hybrid systems, three-hybrid systems, derivatives thereof, etc. The peptide may be larger or smaller than one hundred amino acids, although smaller peptides are preferred in some embodiments. The peptide includes an amino acid sequence selected so that binding of the ligand to the ligand binding domain causes a change in the fusion protein, and that change regulates binding of the interaction domain to the target biomolecule. The peptide is also bonded to the interaction domain at a position selected to permit that change upon ligand binding.

In preferred aspects of the invention, the interaction domains, ligand binding domains, and other detection domains are modular. Each domain may be selected separately, improved separately, redesigned separately, and combined with other selected domains. For example, a domain that changes its conformation in response to taxol binding can be combined with any of a number of potential interaction domains to create a family of taxol-responsive engineered chimeric proteins that bind to different target biomolecules in a manner modulated by taxol. Similarly, if a DNA-binding protein can be regulated by taxol if a taxol-binding domain is attached at a particular (permissive) location, the DNA-binding protein can be regulated by other stimuli by substituting other stimulus-responsive domains that behave similarly to the taxol-binding domain. This “mix-and-match” approach simplifies the design process and multiplies the number of tools available to the biological engineer.

The engineered chimeric protein can be engineered to bind to a DNA sequence (e.g. a promoter, enhancer, etc.) operably linked to a target gene whose expression is then regulated by the inducible change in the engineered chimeric protein. Alternatively, the target biomolecule may be a protein capable of modulating transcription of a target gene, and the change in the engineered chimeric protein may thereby modulate transcription of the target gene. For example, the target biomolecule may be a transmembrane receptor or other protein participating in a signal transduction pathway. In another embodiment, if the engineered chimeric protein has an activity (e.g. DNA binding, protein binding, enzymatic activity, etc.) that is dependent on dimerization, the ligand or other stimulus may modulate dimerization of the protein.

In one preferred embodiment, the engineered chimeric protein includes an interaction domain that binds to a target that is a DNA sequence operably linked to a selected gene to regulate its expression, and a detection domain including a peptide that recognizes a stimulus (e.g. a ligand, a change in a thermodynamic state, etc.). The stimulus causes a change in the engineered chimeric protein, which in turn regulates binding to the DNA sequence and, thereby, expression of the selected gene. The peptide is preferably no more than one hundred amino acids long, and is more preferably shorter (e.g. no more than eighty, no more than sixty, no more than forty, or no more than twenty amino acids long). The change in the engineered chimeric protein may affect DNA binding directly (e.g. by changing the interaction domain) or indirectly (e.g. by regulating dimerization of the engineered chimeric protein, if applicable). The interaction domain may include, for example, a helix-turn-helix motif, as in lambda repressor, a zinc finger motif, as in mammalian steroid receptors, or other DNA binding motifs.

In another preferred embodiment, the peptide that recognizes a stimulus is a ligand binding peptide. Ligand binding causes a change in the engineered chimeric protein, which in turn regulates binding to the DNA sequence and, thereby, expression of the selected gene. The peptide is selected using information from a recombinant display technique. The peptide is preferably smaller than one hundred amino acids. The change in the engineered chimeric protein may affect DNA binding directly (e.g. by changing the interaction domain) or indirectly (e.g. by regulating dimerization of the engineered chimeric protein, if applicable). The interaction domain may include, for example, a helix-turn-helix motif, as in lambda repressor, a zinc finger motif, as in mammalian steroid receptors, or other DNA binding motifs.

Nucleic acids encoding the engineered chimeric proteins of the invention are particularly useful for directing the synthesis of the proteins within a cell. For example, a nucleic acid that includes a promoter directing transcription of an RNA encoding an engineered chimeric protein may be provided to a cell using a plasmid or a virus as a delivery vehicle using method known per se. The resulting engineered chimeric protein can be used within the cell to detect and respond to a stimulus of choice, or may be purified from the cell for use elsewhere.

Engineered stimulus-responsive chimeric proteins of the invention can be used to construct sensor cells that respond to the presentation of a ligand to the engineered chimeric protein. As used herein, “sensor cell” refers to a cell capable of detecting an event or condition and responding in a detectable way. The event or condition may be the stimulus to which the engineered chimeric protein is responsive. For example, the event may be “exposure of the cell to ligand X.” If the engineered stimulus-responsive chimeric protein is a transmembrane receptor, ligand X may bind an extracellular detection domain on the engineered chimeric protein, modulating activity of the engineered chimeric protein. Alternatively, if the engineered chimeric protein is intracellular, ligand X may penetrate the cell; ligand X may, for example, be soluble in the lipids of the cell membrane, or may be transported by a protein in the cell membrane. In an alternative embodiment, the event or condition is not the stimulus, but induces exposure of the engineered chimeric protein to the stimulus. For example, ligand X may bind a receptor that induces an intracellular signaling cascade, inducing synthesis of a second ligand that binds to the detection domain of the engineered chimeric protein.

Sensor cells are useful in monitoring biological, biochemical, chemical, and physical processes and in the construction of engineered cellular machines. Generally, a sensor cell includes at least the engineered chimeric protein, the target biomolecule that binds to the interaction domain of the engineered chimeric protein, and a reporter gene regulated by the target biomolecule whose expression has an effect detectable outside the sensor cell. As used herein, “reporter gene” refers to any gene whose expression has an effect detectable outside the cell. The reporter gene may, for example, alter the viability or fecundity of a cell, may cause it to change color or shape, may induce fluorescence, may induce secretion of a detectable molecule (such as an enzyme or a growth factor), etc, and the effect may be direct (e.g. if the gene product fluoresces) or indirect (e.g. if the gene product is a transcription factor that controls expression of a fluorescent protein).

In one preferred sensor cell, the target biomolecule in the sensor cell is a DNA sequence operably linked to the reporter gene. The change in the engineered chimeric protein upon ligand binding modulates transcription of the reporter gene, permitting indirect detection from outside the sensor cell of a stimulus received inside the sensor cell.

In another aspect, the invention provides an engineered bistable genetic switch. The switch is disposed within a cell or suitable acellular system and comprises a promoter operably linked to an “output gene,” that is, a gene having an expression product that itself is detectable outside the cell, or induces some biochemical change that is detectable as an output of the cell. First and second proteins, at least one of which is a stimulus responsive protein having a structure in accordance with the constructs disclosed herein, respectively modulate transcription of first and second genes to produce first and second translation products. The translation products have, directly or indirectly, opposing effects on the activity of the promoter. Thus, for example, if the system is engineered such that in the presence of a ligand, the output is “on,” then the ligand may effect repression of the first translation product of the first gene, a repressor of the output gene, and the output gene is freely expressed by its promoter to maintain the output in the “on” state. Furthermore, to assure that this state is enduring, the second gene may be engineered to be active to express a repressor of the first gene, or to express an activator of the promoter of the output gene. Conversely, in the absence of the ligand, repression of the first gene does not occur, and its expression product serves to repress expression of the output gene by turning off its promoter. Furthermore, this “off” state may be maintained by a feedback loop, wherein the expression product of the first gene also represses expression of the second gene thereby to shut down expression of the output gene activator, or alternatively, to shut down expression of a repressor for the first gene, both of which avoid stochastic expression of the output gene when it is intended to be in the “off” state. Thus, in a preferred embodiment, the first translation product of the first gene may suppress the level or activity of the second gene, and the second translation product may repress the level or activity of the first gene.

Another embodiment of the bistable switch comprises a cell containing a promoter operably linked to an output gene, the expression of which is detectable as an output of the cell, but in this case the promoter comprises mutually exclusive binding sites for a pair of expression modulating proteins, at least one of which is an engineered chimeric protein as disclosed herein. In this case, for example, in the presence of a stimulus such as a ligand, a stimulus responsive activator protein binds to the promoter of the output gene to activate expression, and the output is “on.” Also, the ligand activates a repressor of a second gene, which encodes and normally expresses a repressor for the output gene, assuring maintenance of the “on” state. Conversely, in the absence of the ligand, the stimulus responsive activator cannot bind to the output gene promoter, and the output is “off.” This state is maintained as the repressor for the second gene also is inactive in the absence of the ligand. The second gene therefore is free to express its repressor, which binds to the second of the mutually exclusive binding sites on the promoter, assuring that it will remain silent.

In still another aspect, the invention may be embodied as an engineered biological logic gate. The gate comprises a cell which includes an output gene, the expression of which defines at least a first state and a second state, e.g., on or off, and is controlled by an expression control DNA, or indirectly by an expression control protein, comprising at least two sites for binding expression modulating proteins constructed in accordance with the invention. The cell also comprises first and second proteins responsive to input stimuli, which proteins bind to one of the binding sites, or modulate expression of another gene product which in turn effects binding to one of the binding sites, thereby to modulate expression of the output gene. Each of the input stimuli responsive proteins have at least a first state and a second state, and the state of the output is determined by the states of the first and second inputs. As will be appreciated by those skilled in the art, the gate may take the form of an AND gate, an OR gate, a NOR gate, or a NAND gate. Such structures may be engineered into cellular or acellular systems wherein the state of the output of a first logic gate determines the state of an input of a second logic gate.

Another form of engineered biological logic gate comprises a cell comprising first and second output genes, the expression of which collectively define an output biochemical activity of the cell, e.g., express the halves of a heterodimeric protein active only when dimerized (to form an AND or NAND gate) or express the same protein from two genes modulated by different stimuli (to form an OR or NOR gate). The genes are controlled by molecules comprising an expression control DNA or an expression control protein. In this case, first and second proteins, each of which bind to the molecule, or modulate expression of another gene product which in turn effects binding to the molecule, modulate expression of the respective output genes. Each of the first and second proteins produce, in response to a biophysical stimulus, at least a first state and a second state of expression of the respective output genes. The output biochemical activity of the cell is dependant on the states of expression of the output genes modulated by the stimuli. At least one of the first and second proteins is a chimeric protein disclosed herein.

Using the engineered chimeric proteins of the invention, logic gates can be designed and combined at will to facilitate the programming of a cell using an algorithm of choice. Such an algorithm could, for instance, be used to engineer a programmable cell for detecting and treating an infection. Such a cell may be programmed, for example, to move randomly until it detects either of two proteins characteristic of a pathogen, at which point the cell emits a signal indicating that an infection has been detected; to emit an antibiotic toxic to the pathogen when and if the cell simultaneously detects both proteins; and to die in response to a chemical injected into the bloodstream by a physician to end the treatment. The modular nature of the engineered chimeric proteins of the invention permits the synthesis of proteins recognizing a variety of stimuli and target biomolecules, permitting the engineering of a multiplicity of logic gates combinable to form complex biological logic circuits.

Because the invention permits modulation of transcription of a gene of choice in response to a stimulus of choice, engineered chimeric proteins of the invention are versatile tools for engineering a multicellular system. For example, a sensor cell as described above can be combined in a multicellular system with a downstream cell that responds to the effect of the reporter gene. For example, a ligand-detection event in the sensor cell can induce a cascade of cell-cell signaling events that modulates cell locomotion, cell viability, cell reproduction, or secretion by one or more downstream cells. Engineered chimeric proteins of the invention are therefore useful in inducing cell patterning and in inducing the patterned deposition of useful molecules on a substrate.

Similarly, a multicellular system may include an upstream trigger cell that responds to a first stimulus by signaling to a cell having an engineered stimulus-responsive protein. The first stimulus may be, for example, a temperature change, electromagnetic radiation, an osmolarity change, or a concentration change of a component such as a nucleic acid, a protein, a hormone, a lipid, or an organic or inorganic compound. The first stimulus induces transmission of a detectable signal to the sensor cell. The detectable signal modulates the exposure of the engineered chimeric protein to a second stimulus that regulates the engineered chimeric protein, thereby modulating expression of a target gene. The second stimulus is preferably a ligand. In response to the detectable signal, the sensor cell may, for example, change the rate of synthesis or degradation of the ligand in the sensor cell or change the location of the ligand in the sensor cell. Alternatively, the detectable signal may itself be a ligand that acts as the second stimulus, in which case the trigger cell may, for example, secrete the ligand into solution or present the ligand on an exterior surface of the trigger cell. A series of interacting trigger cells and sensor cells may be combined to induce a complex cascade of events in response to one or more triggering events, as in a ring oscillator system, for example. Such a cascade is preferably regulated by a biological logic circuit as discussed above.

In another aspect, the invention relates to methods of engineering a ligand-responsive engineered chimeric protein construct. In one embodiment, a recombinant display technique (e.g. phage display, single chain antibody display, retroviral display, bacterial surface display, yeast surface display, ribosome display, two-hybrid systems, three-hybrid systems, derivatives thereof, etc.) is used to identify one or more amino acid sequences of a peptide that bind a preselected ligand. That peptide may be used as the ligand binder in the fusion protein, or alternatively, another peptide may be designed to improve ligand-responsive function based on the amino acid sequence of the starting peptide, or on a consensus sequence derived from the amino acid sequence. The peptide is preferably, although not necessarily, no more than one hundred amino acids in length. An interaction domain capable of binding a target biomolecule is selected (e.g. from the literature), and a potentially permissive position or positions are identified (e.g. using three-dimensional structural data or mutational data) within or adjacent the domain at which insertion of a heterologous peptide may modulate binding of the interaction domain to the target biomolecule. Finally, a construct or, more typically, a plurality of different constructs, having one or more differing forms of the engineered peptide fused to the interaction domain at one or more potentially permissive positions are synthesized and tested to produce a construct in which ligand binding causes a change in the protein, regulating binding of the interaction domain to the target biomolecule.

In another embodiment, one or a plurality of stimulus-receiving peptides that recognize a preselected stimulus are identified. The peptide is no more than one hundred amino acids long, and preferably is shorter. An interaction domain capable of binding a target biomolecule is selected (e.g. from the literature), and one or more potentially permissive positions are identified (e.g. using three-dimensional structural data or mutational data) within or adjacent the domain at which insertion of a heterologous peptide is suspected to permit modulation of binding of the interaction domain to the target biomolecule. A construct or, more typically, a plurality of different constructs having one or more differing forms of the stimulus-receiving peptide fused to the interaction domain at one or more potentially permissive positions are synthesized and tested to produce a construct in which ligand binding causes a change in the protein, regulating binding of the interaction domain to the target biomolecule.

Often, a protein or peptide that recognizes a preselected stimulus can be identified using existing biological knowledge in combination with information in a biological sequence database using modern bioinformatics technology. Accordingly, in one embodiment, information indicative of a stimulus-receiving protein is identified in a database. A permissive position within or adjacent a selected interaction domain is identified, at which insertion of a heterologous peptide permits binding of the interaction domain to its target biomolecule. A construct including the stimulus-receiving protein, or a derivative thereof, fused to the interaction domain at the permissive position is then synthesized and preferably tested for its ability to bind the target biomolecule in a manner modulated by the preselected stimulus.

To test candidate engineered stimulus-responsive chimeric proteins, members of a library of nucleic acids encoding chimeric proteins including a detection domain that recognizes a stimulus and an interaction domain are introduced into cells. The cells include a target biomolecule that binds to the interaction domain of the engineered chimeric protein(s) and a reporter gene whose expression has an effect detectable outside the cell. The target biomolecule may be a nucleic acid operably linked to the reporter gene or a protein capable of modulating transcription of the reporter gene. The cells are maintained under conditions permitting expression of the engineered chimeric proteins encoded by the nucleic acids. The cells are exposed to the stimulus and a cell is identified in which expression of the reporter gene is modulated by the stimulus. A nucleic acid encoding the engineered stimulus-responsive chimeric protein is then isolated from the cell (e.g. after isolation and reproduction of the cell).

The methods for engineering an engineered stimulus-responsive chimeric protein are preferably performed iteratively to further improve the performance of the proteins. For example, after an engineered stimulus-responsive chimeric protein has been identified, a biased library of nucleic acids encoding variations on the engineered stimulus-responsive chimeric protein may be generated. Members of the library are selected or screened for improved sensitivity to the stimulus, improved selectivity for the stimulus, improved speed of switching between the active and inactive states, improved affinity for the interaction domain, greater affinity differences for the interaction domain in the presence and absence of the stimulus, etc. The techniques that permit the intelligent engineering of an engineered stimulus-responsive chimeric protein also facilitate its continued refinement until a tool of the desired precision, specificity and speed has been designed.

The invention also relates to methods exploiting the use of the engineered stimulus-responsive chimeric proteins disclosed herein. In one embodiment, the invention provides a method of detecting a molecule (e.g. a contaminant, an etiologic agent, a product of a fermentation or chemical process, etc.) in a solution by exposing a sensor cell to the solution. For example, various organic compounds known to cause autoimmune disease sometimes contaminate pharmaceutical and feed grades of L-tryptophan manufactured using a fermentation process (see, e.g., Simat et al., Adv. Exp. Med. Biol. 467:469-480). A sensor cell may be used to detect the presence of the contaminant. Alternatively, the molecule may be an etiologic agent such as a biowarfare agent; the sensor cell would thus provide early detection or confirmation of a bioterrorism attack or other biowarfare threat, well before any symptomatic response.

The sensor cell includes an engineered stimulus-responsive chimeric protein and a DNA sequence that binds to the interaction domain of the engineered chimeric protein. The DNA sequence is operably linked to a reporter gene whose expression has an effect detectable outside the sensor cell. The concentration of the molecule (e.g. the contaminant) in the solution modulates exposure of the engineered chimeric protein to the stimulus; in one embodiment, the molecule is the stimulus and binds the detection domain of the engineered stimulus-responsive chimeric protein. The effect of expression of the reporter gene is detected and provides information regarding the presence or concentration of the molecule in the solution.

The engineered chimeric proteins of the invention are also useful in detecting diseases and other disorders, as well as in other diagnostic and prognostic applications. In one embodiment, a sensor cell is administered to a patient; presence of the disease (e.g. prostate cancer) in the patient modulates exposure of the engineered chimeric protein to the preselected ligand (e.g. prostate specific antigen) or other stimulus causing the change in the engineered chimeric protein. The effect of expression of the reporter gene is then detected, thereby permitting detection of the disease in the patient. In another embodiment, a sensor cell is combined with a sample from the patient. The presence in the sample of a disease marker (e.g. prostate specific antigen) indicative of the disease modulates exposure of the engineered chimeric protein to the stimulus. Detecting the effect of expression of the reporter gene is indicative of the presence or absence of the disease marker in the sample.

Similarly, the invention is useful for treating a patient. In one embodiment, a sensor cell is administered to a patient. Exposure of the engineered chimeric protein to the stimulus is modulated by the presence of an abnormal state near the sensor cell. The reporter gene is then expressed, reducing a danger associated with the abnormal state. For example, if the abnormal state is a malignant or premalignant cell, expression of the reporter gene in the sensor cell may reduce the viability or fecundity of the malignant or premalignant cell. If the abnormal state is a protein plaque associated with a disease, expression of the reporter gene may expose the protein plaque to an enzyme that attacks the protein plaque. If the abnormal state is an etiologic agent, a chemical or biochemical species that renders the etiologic agent less harmful (e.g. by killing, digesting, or encapsulating it) may be released.

The invention facilitates the application of pharmacogenomics by facilitating the detection of biomolecules. As used herein, “pharmacogenomics” refers to the study of how genetic variation and resulting phenotypic variation determines a patient's response to a drug. A particular patient's genetic makeup can affect drug responsiveness in at least two ways. A particular variation can render a patient more or less vulnerable to a disease and/or more or less susceptible to responding positively to a drug of choice. Engineered stimulus-responsive chimeric proteins can be used to predict vulnerability and/or a pre-disposition treatment by first detecting the presence of a cellular marker recognizable by the engineered chimeric protein. The cellular marker may, for example, be a protein, peptide, lipid, nucleic acid, carbohydrate, or other organic or inorganic molecule, such as a metabolite, etc. Second, a patient's ability to respond to a drug can be monitored and qualitatively assessed using an engineered chimeric protein responsive to a particular marker.

The invention also provides methods for screening drug candidates that target a particular biochemical pathway. A sensor cell is engineered such that exposure of the stimulus-responsive protein to the preselected stimulus is modulated by activity of the biochemical pathway. The concentration of a drug candidate in contact with the sensor cell is changed; a change in the expression of the reporter gene indicates that the drug candidate indeed modulates the activity of the targeted biochemical pathway.

The invention also facilitates screening a library of nucleic acids (e.g. genes) for those that encode a molecule (e.g. a protein) with a desired biochemical activity. Members of the library are introduced into sensor cells designed such that the biochemical activity itself produces the preselected stimulus or otherwise modulates exposure of the engineered chimeric protein to the stimulus. The cells are maintained under conditions permitting expression of the molecules encoded by the nucleic acids, and a cell expressing the reporter gene at a level indicative of the presence of the desired biochemical activity is identified. The nucleic acid encoding the molecule having the desired biochemical activity is isolated from the cell.

The invention may be used to pattern a biological system. In one embodiment, a sensor cell is maintained under conditions permitting expression of the engineered chimeric protein and is exposed to a position-dependent stimulus, such as a concentration gradient of ligand. Thus, the sufficiency of ligand to modulate expression of the reporter gene varies in a position-dependent manner, causing position-dependent modulation of the reporter gene. If the reporter gene modulates cell movement, the position of the cell will be regulated in response to the concentration gradient. If the reporter gene induces localized deposition of a compound on a substrate, the deposition will be patterned based on the pattern of the ligand concentration gradient.

### BRIEF DESCRIPTION OF THE DRAWINGS

FIGS. 1A-1C are schematic depictions of engineered chimeric proteins of the invention. FIG. 1A depicts engineered chimeric proteins in the presence and absence of a stimulus. FIG. 1B depicts engineered chimeric proteins having an increased affinity for a target biomolecule in the presence of a stimulus. FIG. 1C depicts engineered chimeric proteins having an increased affinity for a target biomolecule in the absence of a stimulus.

FIG. 2 shows the structure of the amino terminal portion of lambda repressor bound to DNA. An arrow indicates a position at which a detection domain may be attached to the protein.

FIG. 3 shows the structure of the DNA binding domain of engrailed bound to DNA. An arrow indicates a potentially permissive position for attaching a detection domain.

FIG. 4 shows the structure of the dimerization domain of lambda repressor. Arrows indicate various potentially permissive positions for attaching a detection domain.

FIG. 5 is a schematic depiction of one embodiment of a simple bistable switch.

FIG. 6 is a schematic depiction of one embodiment of a “flip-flop.”

FIGS. 7A-7E are schematic depictions of simple embodiments of logical gates. 7A depicts a NOR gate. 7B depicts a NOT gate. 7C depicts an AND gate. 7D depicts an OR gate. 7E depicts a NAND gate.

FIG. 8 depicts a NOR gate whose output serves as an input for a NOT gate.

FIG. 9 depicts an exemplary biological logic circuit.

FIG. 10 depicts a signaling pathway regulating a flagellum.

### DETAILED DESCRIPTION OF THE INVENTION

The engineering of novel molecular machines provides precision tools to selectively detect and or modify properties of microenvironments and macroenvironments. If, like a computer, a molecular machine can be programmed in ways limited only by the imagination and skill of the programmer, the tasks a molecular machine can perform are nearly unlimited, ranging from medicine and forensics to environmental engineering, computation, molecular analysis and patterned nanomolecular synthesis. Even a simple program, such as “express protein X in response to stimulus Y,” requires engineering the cell to contain a molecule that not only binds or otherwise recognizes stimulus Y, but that alters the expression of protein X in response. Biological engineering of any magnitude therefore requires a library of molecules capable of recognizing any of a wide variety of stimuli and responding by modulating a chosen biological or biochemical process.

It has been discovered that, by harnessing modular design principles and applying them to biological engineering, engineered chimeric proteins can be designed to be responsive to any of a variety of single or combinations of preselected stimuli. Thus, much like current antibody technology permits the reliable preparation of antibodies that can bind to a preselected epitope, a cell can now be engineered to react to a stimulus of choice. Broadly, the engineered chimeric proteins include a detection domain that recognizes a stimulus and an interaction domain that binds to a target biomolecule.

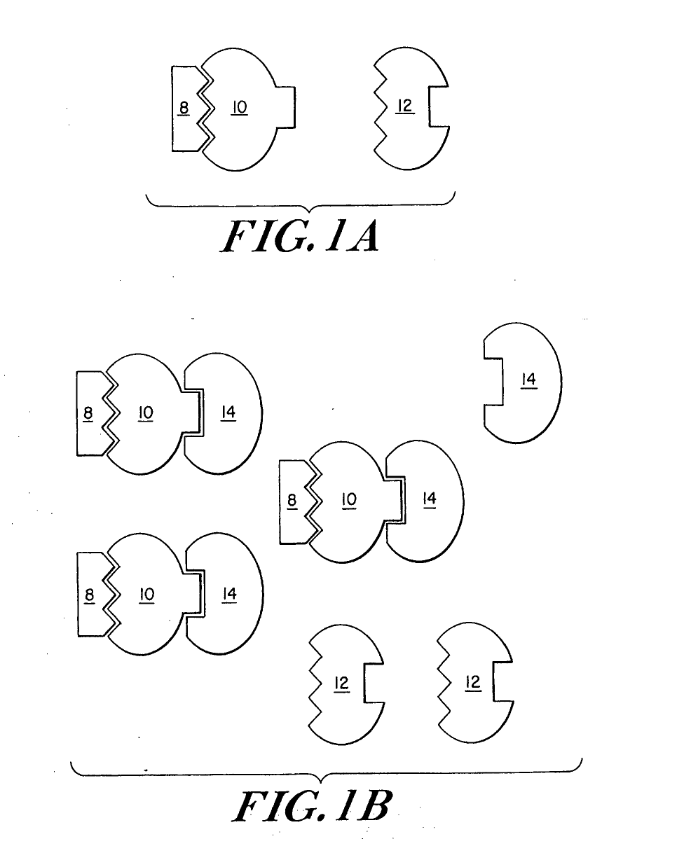

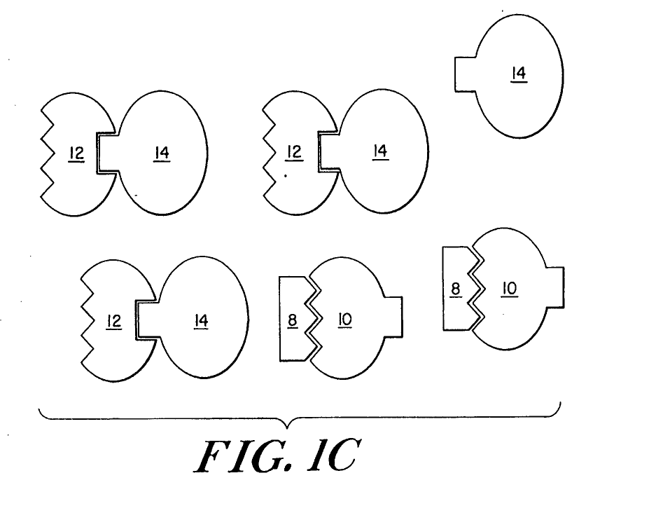

An engineered chimeric protein of the invention has (at least) two states characterized by the presence or absence of a preselected stimulus. Referring to FIG. 1A, the engineered chimeric protein exists in a first state 10 in the presence of stimulus 8, and in a second state 12 in the absence of stimulus 8. Although first state 10 is depicted using a shape different from that of second state 12, the engineered chimeric protein need not have a detectably different shape in the first and second states, although it often does. Regardless of the presence or absence of a detectable shape change, the conversion of the engineered chimeric protein between first state 10 and second state 12 regulates the interaction of the engineered chimeric protein with a target biomolecule 14. As shown in FIG. 1B, bound state 10 may have a higher affinity for target biomolecule 14 than does unbound state 12, or, as shown in FIG. 1C, the opposite may be true.

The versatility of the invention is provided to a significant extent by the modularity of the detection and interaction domains. Thus, a detection domain that recognizes a preselected stimulus can be selected and bound to a chosen interaction domain. This “mix-and-match” ability permits the skilled artisan to regulate a biological pathway of choice using a ligand of choice, once suitable interaction domains and detection domains have been identified.

I. Stimuli

The engineered chimeric proteins of the invention detect and respond to a preselected biophysical stimulus. Generally, the chosen stimulus may be any event or condition capable of directly or indirectly modifying the state or activity of a protein. In a preferred embodiment, the stimulus is a ligand that physically interacts with the protein. The ligand may, for example, be an organic molecule such as a biomolecule or synthetic chemical, an inorganic molecule such as an ion, or an electron. Alternatively, the stimulus may be a change in a thermodynamic state, such as pressure (including osmotic pressure), temperature, etc., a change in electromagnetic radiation (e.g. a pulse of light, a decrease in light intensity, or a change in wavelength), or other detectable change.

II. Detection Domains

The detection domain includes a peptide that recognizes a stimulus. The peptide may include natural and/or nonnatural amino acids, and may be posttranslationally modified. Many natural detection domains are known and may be used to inform the selection of a detection domain or peptide for engineering into an engineered stimulus-responsive chimeric protein. In some embodiments, the peptide is preferably not unduly large, and is preferably no more than one hundred amino acids in length, and may be significantly smaller.

The nature of the detection domain may vary based on the nature of the desired stimulus. If the stimulus is a ligand, the ligand binds to the detection domain (alternatively referred to as a ligand binding domain). The detection domain is preferably known to alter its conformation in response to a ligand binding event: such a conformational change may then be communicated to a contacting interaction domain. If the stimulus is a temperature change, the detection domain may be derived from a known temperature sensitive protein or may be derived from a genetic selection or screen for peptides that undergo a conformational change in response to a temperature change. If the stimulus includes light, the detection domain may be derived from a known light-responsive protein, may be derived from a genetic selection or screen, and/or may be posttranslationally modified to incorporate a chemical complex that converts light energy to other forms of energy. For example, a peptide may be modified to incorporate a ruthenium complex that emits an electron in response to light; the electron may then modify the activity of an attached protein (see, e.g., Bjerrum et al., J. Bioenerg. Biomembr. 27(3):295-302). Alternatively, a gold nanocrystal may be posttranslationally attached to a peptide. The gold nanocrystal absorbs radio waves, locally heating an associated protein.

In embodiments in which the stimulus is a ligand, a recombinant display technique may be used to identify candidate peptides. Useful recombinant display techniques include, but are not limited to, phage display (see Hoogenboom et al., Immunol Today 2000 August; 21(8):371-8), single chain antibody display (see Daugherty et al., Protein Eng 1999 July; 12(7):613-21; Makeyev et al., FEBS Lett 1999 Feb. 12; 444(2-3):177-80), retroviral display (see Kayman et al., J Virol 1999 March; 73(3):1802-8), bacterial surface display (see Earhart, Methods Enzymol 2000; 326:506-16), yeast surface display (see Shusta et al., Curr Opin Biotechnol 1999 April; 10(2):117-22), ribosome display (see Schaffitzel et al., J Immunol Methods 1999 Dec. 10; 231(1-2):119-35), two-hybrid systems (see, e.g., U.S. Pat. Nos. 5,580,736 and 5,955,280), three-hybrid systems, and derivatives thereof. Recombinant display techniques identify peptides capable of binding proteins, small molecules, and inorganic ligands (see, for example, Baca et al., Proc Natl Acad Sci USA 1997 Sep. 16; 94(19):10063-8; Katz, Biomol Eng 1999 Dec. 31; 16(1-4):57-65; Han et al., J Biol Chem 2000 May 19; 275(20):14979-84; Whaley et al., Nature 2000 Jun. 8; 405(6787):665-8; Fuh et al., J Biol Chem 2000 Jul. 14; 275(28):21486-91; Joung et al., Proc Natl Acad Sci USA 2000 Jun. 20; 97(13):7382-7; Giannattasio et al., Antimicrob Agents Chemother 2000 July; 44(7):1961-3). Using phage display, for example, a ligand binding peptide may be selected by: immobilizing a chemical to a surface, passing the combinatorial phage mixture over the surface, washing to remove non-binding moieties, collecting the attached phage, amplifying the phage in an appropriate naïve host, then performing this procedure of selection iteratively until one or more strong, high specificity binding epitopes are obtained.

The epitopes are preferably selected from a library of random or biased sequences that may or may not be disulfide constrained. A biased library has randomized positions interspersed with conserved positions. A disulfide constrained sequence (constrained by the existence of a disulfide bond) often more efficiently binds to ligands and is more likely to be modular and to maintain its binding capacity when imported into a new protein.

For example, peptides may be selected which will bind specifically to phenylalanine. Specific binding peptides may be derived from a library of linear or cysteine-constrained peptides presented on bacteriophage surfaces. Phenylalanine binding epitopes may be selected in the following way: a combinatorial phage library is contacted first with agarose beads (to remove epitopes that bind to agarose), then with tyrosine-agarose beads (to remove epitopes that bind to tyrosine, which is structurally very similar to phenylalanine), and finally with phenylalanine-agarose beads (to isolate those epitopes that do bind to phenylalanine but not to agarose or tyrosine). Several rounds of selection and amplification in this manner result in the isolation of phages bearing epitopes that bind specifically to phenylalanine.

A peptide selected using a recombinant display technique may be used to engineer an engineered ligand-responsive chimeric protein. Alternatively, information from the selected peptides may be used to design the ligand binding domain. For example, a particular pattern of amino acids may be present in a number of peptides selected using a recombinant display technique. That pattern, or a variation on the pattern, may be used to design a small ligand binding peptide for use in the engineered ligand-responsive chimeric protein. Thus, the actual ligand binding peptide used does not necessarily correspond to any single peptide from the recombinant display technique. To develop sequences with further enhanced characteristics, one or more amino acids in a peptide from the recombinant display technique may be mutated in a random or systematic fashion and tested for activity, using, for example, the agarose bead technique described above, or using any of the other well-known methods for detecting a binding interaction.

Preferred detection domains incorporate one or more features designed to facilitate their function in an engineered stimulus-responsive chimeric protein and to promote allosteric changes in the engineered chimeric protein in response to the stimulus. In a biased library, the features are preferably incorporated by using conserved residues that confer the features on the detection domain. For example, the detection domain may preferably be designed to present a hydrophobic surface in response to the stimulus. Hydrophobic interactions are important factors in protein folding and useful in magnifying the structural effects of a detection event such as a ligand binding event. The binding surface of a small molecule to a protein is often about one hundred to two hundred square angstroms, and the binding energy between them rather small (e.g. less than fifty to one hundred kilocalories per mole. Protein-protein interfaces often span about one to two thousand square angstroms, with commensurate binding energies. Thus, leveraging a small binding event into a large hydrophobic change in the protein structure allows the engineering of a more robust structural response to the ligand binding event. The process for engineering an interaction domain to respond to a stimulus is further described in section IV, below.

The detection domain may be designed to adopt a predominantly amphipathic structure upon ligand binding. Amphipathic helices are generally more soluble and less prone to aggregation than non-amphipathic structures. Furthermore, with an amphipathic structure, a small perturbation in the structure is sufficient to create a hydrophobic patch useful for interacting with a stimulus such as a ligand, or for transmitting the effects of a detection event to the rest of the protein.

Ideally, molecular modeling programs and tools known in the art are used to analyze the conformation of the detection domain in the presence and absence of the stimulus to identify conformational changes that can be harnessed to induce an allosteric change in an interaction domain. This modeling at least includes the detection domain in the presence and absence of the stimulus, and preferably also analyzes the structure of an attached interaction domain. Again, conserved amino acids used in a biased library for identifying candidate stimulus-receiving peptides are preferably selected to confer a specific statistical ensemble structure upon recognition of the stimulus to facilitate allosteric effects on the engineered chimeric protein.

III. Interaction Domains

The interaction domain binds to a target biomolecule in a manner conditioned upon either the presence or absence of recognition of a stimulus by the detection domain. The target biomolecule is often a DNA, RNA, or a protein, but may be a different biomolecule, such as a carbohydrate, lipid, etc. The interaction domain is often derived from a naturally-occurring nucleic acid binding or protein binding domain. Alternatively, the interaction domain may have no natural counterpart, but be designed using molecular modeling tools or be derived from screening a randomized library.

In one embodiment, the interaction domain is preferably a DNA binding domain. Suitable DNA binding domains include those derived from natural proteins including, for example, bacterial proteins (e.g. alpA, araC, arsR, asnC, birA, lambda repressor, cro, crp, deoR, dtxR, fis, fur, gntR, hipB, iclR, lacI, lexA, luxR, lysR, marR, merR, modE, mor, ner, ntrC, pin, rpoD, rpoN, sor C, tetR, trpR, ompR, toxR, cspA, ihf, metJ, mnt, traY, dksA, abrB, argR, dps, int, hha, hns, intR, dnaJ, mod, mtlR, glpG, bolA, nagC, papB, papI, rop, rtp, tus, etc.), yeast proteins (e.g. PHO4, MATalpha2, GCN4, GAL4, etc.), plant proteins, insect proteins (e.g. engrailed, antennapedia, etc.), fish proteins, bird proteins, and mammalian proteins (e.g. HMG-I, STAT-1, NFkappaB p65, c-myb, TBP, c-myc, max, E2F-1, DP-1, fos, jun, p53, Oct-1, glucocorticoid receptor, pit-1, etc.).

The interaction domain should be modular. It is important that the interaction domain function as a discrete entity that can be fused to a protein having one or more other domains, conferring on the engineered chimeric protein an ability to bind to a target biomolecule of interest. This modular characteristic facilitates the construction of entire families of engineered stimulus-responsive chimeric proteins, such that an interaction domain can be made responsive to a stimulus of choice. Conveniently, natural protein binding domains and DNA binding domains have routinely been shown to be modular. Indeed, this modularity is the basis for two-hybrid screens, in which a DNA binding domain is fused to a bait protein of choice to screen for other proteins that interact with the bait protein; suitable DNA binding domains for these screens are known to include, for example, the DNA binding domains of LexA, ACE 1 (CUP 1), lambda repressor (also known as lambda cI), lac repressor, and GCN4 (see U.S. Pat. No. 5,580,736 to Brent et al.). Naturally existing protein binding domains have also been shown to be modular. Lambda repressor, for example, has a DNA binding domain and a dimerization domain, as does the yeast GCN4 protein. The dimerization domain of lambda repressor can be completely removed from the protein and replaced with the dimerization domain of GCN4. In this chimeric protein, the GCN4 dimerization works normally, promoting dimerization of the chimeric protein. The DNA binding domain of lambda is also modular and promotes binding to DNA even when combined with the “foreign” GCN4 dimerization domain.

If a derivative of a naturally occurring interaction domain is used, the interaction domain may be modified to interact with a different target biomolecule. For example, U.S. Pat. No. 5,789,538 to Rebay et al. discloses how to modify Zif268, a natural DNA binding protein with “zinc finger” motifs, to create a protein with a DNA binding specificity different from that of any known zinc finger protein. At least one amino acid that contacts the DNA is replaced with a different amino acid at the same position. The base sequence specificity of the resulting protein is determined by selecting the optimal binding site from a pool of duplex DNA with random sequence.

IV. Positioning the Detection Domain with Respect to the Interaction Domain

The detection domain is bonded to the interaction domain at a position that causes the binding of the interaction domain to a target biomolecule to be conditional on the presence or absence of a stimulus. Accordingly, the position at which the detection domain is placed must be at least somewhat tolerant: if the presence of the detection domain too greatly disrupts the structure of the interaction domain, binding to the target biomolecule may be lost regardless of the presence or absence of the stimulus. Functional data about tolerant positions in and about the interaction domain and structural data about the interaction domain and its contacts with a target biomolecule are generally very informative regarding proper placement of the detection domain.

A. Structural Data

Structural data are very useful in the correct placement of engineered insertions, deletions and mutations in proteins. High resolution (1-2 Å) crystal structure and NMR of known proteins and their domains are the most definitive determinants of protein architecture known today and medium resolution (2-5 Å) are also useful. SWISSPROT, PDB, Pfam and other structure databases are repositories for an increasing number of protein family, fold and function representatives. Even if the precise structure of the interaction domain has not been determined, structural data about the interaction domain can generally be inferred from structural data of other domains that are more than thirty percent identical to the interaction domain. Using a technique known as “threading,” the sequence of the interaction domain is algorithmically substituted for the sequence of the domain with known structure; amino acids that are conserved between the two domains are presumed to occupy similar positions in the structure. The result is an inferred structure of reasonable integrity; higher degrees of homology between the interaction domain and the domain of known structure result in increasing reliability of the inferred structure.

From structural information, candidate positions for the detection domain are identified. For example, proteins generally consist of alpha-helices and beta-sheets joined by segments often referred to as loops or turns. In many instances, insertion of a heterologous peptide directly into an alpha-helix or a beta-sheet will prevent the proper folding of that structure. Accordingly, loops and turns are preferred candidate locations for insertion of a heterologous peptide. Structural data may also suggest that a location may be less desirable for other reasons. For example, inserting an amino acid at a particular position may sterically interfere with other amino acids in the structure, may disrupt important hydrophobic-hydrophobic or ionic interactions, or form inappropriate interactions with other portions of the structure. Of course, some disruptions are acceptable, or even desirable: a disruption that can be “undone” by a stimulus provides an engineered ligand-responsive chimeric protein that only binds a target biomolecule in the presence of the stimulus. Nevertheless, major disruptions are, in most instances, preferably avoided, as they are less likely to be reversible upon addition or removal of ligand.

B. Functional Data

Functional data showing which positions of the interaction domain are important for binding to the target biomolecule are also very useful in identifying candidate positions for inserting a detection domain. Among the most useful data in this regard are data showing which positions in the detection domain actually tolerate insertions. An interaction domain can be scanned for tolerant positions by transposon mediated random insertions into the interaction domain using a system such as the GPS-LS linker scanning system from New England Biolabs, which uses a Tn7 based transposon and restriction digests to insert 15 nucleotides at random positions in the nucleic acid. Thus, interaction domains with insertions of five amino acids at random positions can be tested for binding to the target biomolecule. If, at a particular position, an insertion of five amino acids does not disrupt binding to the target biomolecule, that position is a preferred candidate position for the detection domain. Alternatively, a combinatorial method (e.g. as described in WO00/72013) can be used to generate libraries of nucleic acids encoding an interaction domain with randomly positioned insertions.

Functional and structural information can also be inferred from studying amino acids that are conserved at a particular position among members of a family of related proteins. Conserved residues can be identified, for example, by performing a multiple sequence alignment of related proteins using programs such as CLUSTALM, CLUSTALK, or CLUSTALW, which are known in the art for this purpose, or by visual inspection using information from databases from, for example, Pfam or SCOP. At a particular position, if the same amino acid occurs in, for example, at least ninety percent of the family members, that amino acid is likely to be relevant to the structure or function of the protein. If genetic alleles in which the activity of the interaction domain is altered are known, one or more of the positions at which the amino acid sequence of that allele differs from the sequence of the wild-type allele is relevant to the structure or function of the protein. Similarly, if a mutation in a related protein is known to affect its activity, and if the mutated amino acid is an amino acid that is conserved between the two proteins, that amino acid is likely important to the structure or function of the interaction domain. If, among related proteins, changes at a first position are routinely accompanied by changes at a second position, the covariance of the amino acids may indicate that the amino acids at those positions interact in a manner relevant to the structure or function of the protein. Generally, locations that do not appear to be critical structure/function regions (i.e. locations that are in loops, locations that are not highly conserved and do not covary, etc.) are preferred candidate locations for binding the detection domain. Critical interfaces (e.g. within the interaction surface between the interaction domain and the target biomolecule) are not preferred positions at which to insert a detection domain, as insertions there are likely to permanently disrupt the function of the interaction domain.

C. Summary of Predictive Design Considerations

Structural data (e.g. from high resolution or medium resolution crystal structures), genetic data and biochemical data can be used to develop a comprehensive structure/function picture of an interaction domain. This comprehensive picture provides the ability to propose sites for insertion, deletion, or mutagenesis. The process for building this comprehensive picture may include any combination of the following discrete steps:

1) Identify and download the protein sequence(s) for the interaction domain using, for example, programs such as FASTA, BLAST, PSI-BLAST, or other tools from, for example, the National Center for Biotechnology Information.

2) Retrieve the crystal structure of the protein(s) from PDB, SWISSPROT, etc.

3) Perform a multiple sequence alignment of all sequences related to the sequence of the interaction domain using a program such as CLUSTALK, CLUSTALM, or CLUSTALW.

4) Using the MSA, identify residues more than ninety percent conserved. These residues are likely to be relevant to the function or structure of the protein.

5) Determine if the protein has genetic variants with differing function. Those positions that differ among the variants are likely to be associated with an altered structure, specificity, or function.

6) Determine the effects of any reported mutations on the activity of the protein or of a related protein. If mutation of a residue has an effect on one of the conserved residues identified above, that residue is likely important to the structure or function of each related protein.

7) Determine the incidence of covariance of residues surrounding the conserved residue positions among related proteins. Covariance can be used to infer a functional relationship between positions in a protein without specific regard to overall sequence as described above.

8) If the structure is not known, then use threading as described above to approximate the structure of the protein. The protein must be more than 25-30% homologous to the comparison structure for the resulting structural prediction to be reliable. If threading is not an option, use sequence alignment and look for covariance as above.

9) Define regions of the protein of interest that do not appear to be involved in critical structure/function areas (i.e. loops and nonconserved, noncovariant areas, etc.).

10) Further define the areas of contact of the protein with itself (if applicable), with the target biomolecule and, if the detection domain binds a preselected ligand, with the ligand. Crystal structure data of the bound and unbound forms of the protein can be used to inform engineering efforts to more accurately place novel, noninterfering detection domains into the protein. The desired insertion point of the detection domain would be at a position that would not interfere with normal function of the interaction domain but would interfere upon recognition of the stimulus by the detection domain.

11) Fuse the sequence of the detection domain into the positions identified above and test for modulation of the function of the interaction domain by the presence or absence of the stimulus. The detection domain may be selected from a sequence library, such as a library of random linear, random disulfide constrained, biased linear, and disulfide constrained sequences. A biased library would have randomized positions interspersed with conserved positions designed to adopt an amphipathic structure and a hydrophobic presentation upon detection of the stimulus. Conserved positions would also be designed to confer a specific statistical ensemble structure upon detection of the stimulus, thereby to engineer an allosteric change responsive to the stimulus.

D. DNA Binding Domains

In a preferred embodiment, the interaction domain is a DNA binding domain. Many modular DNA binding domains have been characterized structurally and functionally, facilitating the identification of candidate locations for a detection domain. Examples include the lac repressor (see Bell et al., Nat. Struct. Biol. 7:209-214; Lewis et al., Science 271:1247-1254; Friedman et al., Science 268:1721-1727; Chuprina et al., J. Mol. Biol. 234:446-462; Bell et al., Curr. Opin. in Struct. Biol. 11:19-25; and Matthews et al., Prog. Nucleic Acid Res. Mol. Biol. 58:127-164), the trp repressor (see Wallqvist et al., Biophys. J. 77:1619-26; Joachimiak et al., Proc Natl Acad Sci USA 80:668-72; Schevitz et al., Nature 317:782-6; Lawson et al., Proteins 3:18-31; and U.S. Pat. No. 5,190,873), purR (see Lu et al., Biochemistry 37:971-82; Glasfeld et al., J. Mol. Biol. 291:347-361; Nagadoi et al., Structure 3:1217-24; Schumacher et al., Science 1994 Nov. 4, 266(5186):763-70; and Arvidson et al., Nat. Struct. Biol. 5:436-41), and ureR (see Poore et al., J. Bacteriol. 183:4526-35; and D'Orazio et al., Mol. Microbiol. 21:643-55).

Conveniently, very similar DNA binding structures are found in many natural DNA binding proteins. At the most basic level, in a DNA-binding domain, an alpha-helix normally makes the contacts with the nucleotide bases permitting the protein to “read” a DNA sequence. Furthermore, specific types and combinations of helices and connecting structures are found in the DNA binding domains of proteins that may otherwise appear to be unrelated. Examples of these structures include the “helix-turn-helix” motif, found in viruses, bacteria, and eukaryotes including mammals, and the “zinc finger” motif. Regardless of the DNA sequence recognized, a given motif binds DNA using a structure that is very much the same. Accordingly, once an engineered ligand-responsive chimeric protein has been designed using one DNA binding domain containing a given motif, the results are rapidly applicable to other DNA binding domains containing similar motifs. Modular engineering principles thus ease the design of engineered ligand-responsive chimeric proteins for a wide variety of DNA binding domains.

D1. Helix-Turn-Helix Motifs

As its name suggests, the helix-turn-helix motif includes two alpha-helices separated by a turn. Both helices contact the DNA; the latter helix is the “recognition” helix, making base-specific contacts that permit the domain to specifically bind a particular DNA sequence. The motif is generally present in a DNA binding domain including other alpha-helices and/or beta sheets that help to present the helix-turn-helix to the DNA and often make additional DNA contacts. The motif has been characterized in the context of many proteins, including viral proteins such as lambda repressor (see Bell et al., (2000) Cell 101(7): 801-811; and Jordan et al., (1988) Science. 242(4880): 893-899), Cro repressor (see J. Mol. Biol. 280:129-36), phage P22 C2 repressor (J. Mol. Biol. 235:1003-20), and phage 434 repressor (see Structure 1:227-240); bacterial proteins such as AraC (see Bustos et al., Proc. Natl. Acad. Sci. USA 90:5638-5642); and eukaryotic proteins such as the homeobox family of proteins (see, for example, J Mol. Biol. 284:351-61).

Lambda Repressor

Lambda repressor binds to DNA as a homodimer. The DNA sequence bound by lambda repressor is relatively symmetrical, and each subunit binds to one half of the symmetrical sequence. The high accuracy crystal structures of the X repressor amino-terminal fragment with and without its DNA operator and of the lambda repressor carboxy-terminal dimerization domain have been determined (see Bell et al., (2000) Cell 101(7): 801-811; and Jordan et al., (1988) Science. 242(4880): 893-899). The identity and characteristics of the domain structures in lambda repressor have been elucidated by the engineering of “domain swapping” experiments. These studies showed that when domains derived from related phages 434 and P22 were exchanged for lambda domains, the chimeric repressor functioned (see Whipple et al. (1994) Genes Dev. 8:1212-1223). It is also possible to replace amino acids in the recognition helices of lambda repressor involved in making contact with operator sequences with those from related repressors: the resultant mutant lambda repressors now bind to operator sequences of those other repressors. The C-terminus dimerization domain of the lambda repressor includes amino acids 132-236 and the N-terminus DNA binding domain includes amino acids 1-92; with the linker region being amino acids 92-132.

Many derivatives of this protein have been made. The structure/function relationship of the X repressor protein is well characterized (see Ptashne, A Genetic Switch: gene control and phase lambda (1986) Cell Press). Chimeric constructs include those that alter the specificity of the interaction of lambda repressor with its operator sequence to direct repressor binding to new sequences or to allow for altered dimerization characteristics (Donner et al., J. Mol. Biol. 283: 931-946).

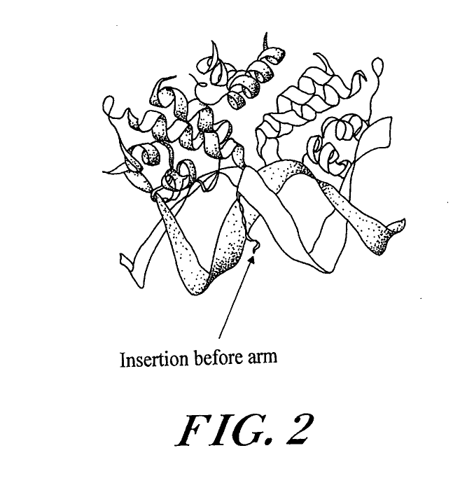

An engineered ligand-responsive protein combining the DNA binding domain of lambda repressor and a heterologous ligand binding domain has been generated and proven effective as discussed in greater detail in the Examples section below. The high resolution (1.8 angstrom) crystal structure of the lambda repressor DNA binding domain identifies and describes a role for the first six amino acids of the DNA binding domain (referred to as the arm) of one monomer unit in contacting the major groove of DNA at the consensus half site (Beamer et al., J. Mol. Biol. 227:177-196). The arm on the other monomer which contacts the non-consensus half lacks electron density and is thus thought to stay disordered. These observations were validated by Kim and Hu (Kim et al., Proc. Natl. Acad. Sci. USA 92:7510-7514) and the importance of these residues in contributing to DNA binding further established. As shown in the examples, addition of a linear peptide at the amino-terminal end does not disrupt the amino acid-DNA contacts: the repressor functions normally despite the presence of the additional peptide sequence. (The three-dimensional structure of the DNA binding domain is shown in FIG. 2, with an arrow pointing to the amino-terminal end of the protein.) Ligand binding, however, disrupts the function of the protein, presumably by reducing the flexibility of the peptide and hindering the interactions with the DNA backbone or contacts between the two arms of the monomers. Importantly, this demonstrates that the affinity of the engineered chimeric protein for the DNA can be regulated by interfering with nonspecific contacts with the DNA backbone, and does not require modification of the core helix-turn-helix residues, which are more likely to be resistant to engineering efforts.

AraC 100781 Helix turn helix motifs are also present in transcriptional activators such as the araC protein. araC is a transcriptional regulator of the L-arabinose operon in E. coli. Functional domains of the protein have been defined: the amino terminal end (aa 1-170) dimerizes the protein and binds the sugar arabinose; the carboxy terminal end (aa 178-292) binds DNA and contacts RNA polymerase (see Bustos et al., Proc. Natl. Acad. Sci. USA 90:5638-5642). The two regions are connected with a linker of at least 5 amino acids (Eustance et al., J. Bact. 178:7025-7030). Both the DNA binding region and dimerization domain retain activity when fused to heterologous domains. Functional hybrids have been reported between the araC DNA binding domain and a leucine zipper dimerization domain derived from C/EBP (Bustos and Schleif, PNAS, 90, 5638-5642). The role of the linker region in araC has been investigated (Eustance et al., J. Mol. Biol. 242:330-338). The araC dimerization domain was linked to the lexA DNA binding domain with the linker region from lambda repressor and the resultant chimera was functional in DNA binding. Moreover altering the linker length permitted modulation of DNA transcription via placement of the DNA binding sites within the promoter. This demonstrates that araC is a truly modular protein.

Based on the similarities between the DNA binding domains of araC and lambda repressor, the “arm” sequence of araC is predicted to be a likely location for a detection domain to generate an engineered stimulus-responsive chimeric protein. Other possible sites for insertion can be identified by, for example, use of the transposon-mediated linker scanning system or of combinatorial libraries as disclosed in PCT publication WO00/72013 to identify permissive positions within the DNA binding domain. Any araC construct can easily be tested for activity by using a reporter construct such as pBAD-lacZ, known in the art to be responsive to araC function.

Eukaryotic Homeobox Proteins

The helix-turn-helix motif is also present in eukaryotic proteins such as homeotic transcription factors. These proteins share a conserved region, known as the homeobox, which is known to be involved in specific binding to DNA. The crystal structures of homeobox domains from engrailed (J Mol. Biol. 284:351-61), Oct-1 (Cell 73:193-205) and Pit-1 (Genes Dev., 11: 198-212) POU domains bound to their cognate DNAs show remarkable similarity to the helix-turn-helix motif, with the exception that the recognition helix is longer. These proteins can function as transcriptional activators or repressors, depending on the other domains and the interaction of the other domains with either co-activators or members of the transcription apparatus. For example, the Oct-1 protein itself does not directly activate transcription, but recruits the acidic activator VP-16 and HCF and it is this complex that is efficient in recruiting RNA polymerase and increasing transcription.

The Engrailed protein in Drosophila melanogaster acts as a transcriptional repressor, regulating the activity of other homeobox genes (Han et al., EMBO J. 12:2723-2733). The carboxy-terminus of the gene contains the conserved homeobox and co-crystal structures with DNA of the wild type homeodomain (J Mol. Biol. 284:351-61) as well as a mutant form (Tucker-Kellogg et al., Structure 5:1047-1054) are available. The structure reveals an extended N-terminal arm and three helices. The third helix (aa 42-57) functions as the recognition helix and binds in the major groove of DNA. A point mutation, Gln50Lys, changes the binding specificity from TAATCC to TAATTA. The N-terminal arm and the recognition helix are involved in both specific contacts with bases and interactions with the sugar phosphate backbone.

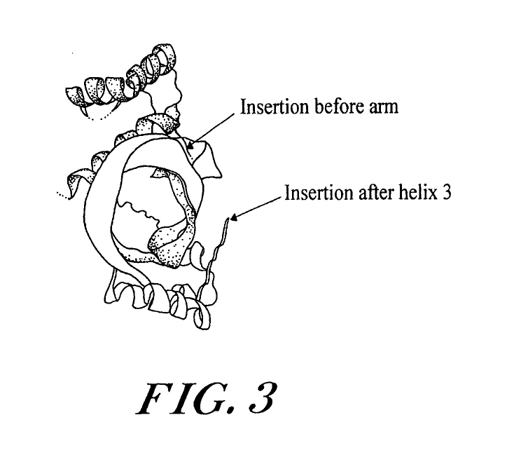

One group (Pan et al., Protein Science, 4, 2279-2288) showed that, in the arm of the engrailed protein, residues 2-6 of the protein do indeed contribute significantly to the binding to DNA. Thus, not only does the DNA binding domain of the homeobox protein have a helix-turn-helix motif much like that of lambda repressor, but the amino terminal residues are similarly important for DNA binding. Accordingly, a position at or very near the amino terminus of a homeobox protein is an excellent candidate location for attaching a detection domain to engineer a stimulus-responsive protein as with lambda repressor. This position is indicated in FIG. 3, showing one view of the structure of the DNA binding domain. Insertion at this site of additional amino acids without strong intrinsic secondary structure is unlikely to destabilize the existing arm-DNA interactions and the resultant protein should still be able to bind DNA. If the stimulus is a ligand, for example, ligand binding may stabilize the unstructured ligand binding domain and interfere with the protein-DNA interaction. Other locations (e.g. following the carboxy-terminal end of the third helix, as shown in FIG. 3) may also be good candidate locations, as insertions are likely to allow proper folding of the protein and binding to DNA, at least in the absence of ligand.

D2. Zinc Finger Motifs

Another common motif involved in DNA binding is the zinc finger domain, which usually occurs in tandem copies. One form of the Zinc finger has a consensus sequence Cys-X2-4-Cys-X3-Phe-X5-Leu-X2-His-X3-His (SEQ ID NO:1, SEQ ID NO: 2 and SEQ ID NO: 3) which forms a “Cys-His” finger. The C-terminal part forms α helices which bind DNA, and the amino terminal part forms beta sheets (Klug et al., Trends Biochem. Sci. 12:464-469). Steroid hormone receptors contain a specialized form of the zinc finger with the consensus sequence Cys-X2-Cys-X13-Cys-X2-Cys (SEQ ID NO: 4)(Evans et al., Cell 52:1-3). Glucocorticoid and estrogen receptors each contain 2 zinc fingers: one controls specificity of DNA binding and the other controls specificity of dimerization. In the estrogen and progesterone DNA binding domains, specific amino acids in the recognition helix and in the flexible linker region between the two zinc fingers are important for DNA binding affinity and specificity (Chusacultanachai et al., J. Biol. Chem. 274:23591-23598). Accordingly, positions within the helices or in the linker are not the best candidate positions for placing a detection domain. On the other hand, the beginning of the first beta sheet that preceding the first zinc finger is likely more tolerant of insertions. Other candidate locations may be identified, for example, by linker scanning mutagenesis as described above.

E. Protein Binding Domains

In some embodiments, the interaction domain is a protein-binding domain, such as a domain required for dimerization or for binding a separate protein.

E1. Dimerization Domains

As with DNA binding domains, dimerization domains are often modular and susceptible to biological engineering. By engineering a dimerization domain to be stimulus-responsive, one can regulate the function of any protein that requires dimerization for activity. Dimerization of a transmembrane receptor, for example, can be rendered dependent on a chosen stimulus. Regulating dimerization of key signal transduction proteins can modulate intracellular signaling pathways. By regulating dimerization of a homodimeric or heterodimeric transcription factor, the interaction of that transcription factor with DNA, RNA polymerase, or other transcription factors can be controlled. Furthermore, dimeric proteins are particularly useful tools for constructing logic gates and circuits. Whereas the activity of a monomeric protein is directly proportional to the percentage of monomers in an active state, the relationship between the activity of a dimeric protein and of its corresponding monomers is exponential. Accordingly, regulation of dimeric proteins can provide signal to noise ratios that are superior to those provided with monomeric proteins.

Importantly, in accordance with modular engineering principles, a stimulus-responsive dimerization domain can be fused to any DNA binding domain of interest—generally a DNA binding domain of a dimeric DNA binding protein. For example, if the dimerization domain of lambda repressor, or GCN4, or AraC, or another dimeric transcription factor is removed and replaced with a stimulus-responsive dimerization domain, the biological activity of that engineered chimeric protein becomes stimulus-responsive. Thus, a single stimulus-responsive dimerization domain can be used repeatedly to render stimulus-responsive an arbitrarily-selected dimeric transcription factor. Indeed, any signaling pathway involving a multimeric protein can be rendered stimulus-responsive by replacing its dimerization domain with a ligand-responsive dimerization domain. The time and expense associated with de novo design is largely avoided through the use of a reusable stimulus-responsive dimerization domain.

Leucine Zippers

One of the most common dimerization modules is the leucine zipper, which is made up of heptad sequences with leucine at every seventh position (see Landchulz et al., Science 240:1759). Each monomer forms an amphipathic helix. The leucine zipper is postulated to form a coiled coil by wrapping of the amphipathic helices around one another, with the leucines becoming located within the hydrophobic interface between monomers (O'Shea et al., Science 243:538). The Saccharomyces cerevisiae activator GCN4 contains, in addition to the basic region that binds to DNA, a leucine zipper, which serves as a dimerization domain, even when used heterologously (see, for example, Hu et al., Science 250:1400-1403). In the case of GCN4, the activator is a homodimer. AP-1 (activator protein 1) is an example of a heterodimeric eukaryotic transcription factor formed by association of Jun and Fos family members (reviewed in Wisdom, Exp. Cell Research 253:180-185). Though both Jun and Fos contain leucine zippers, only Jun can homodimerize, with heterodimerization between Jun and Fos being favored over homodimerization. Leucine zippers have also been postulated to dimerize in the transmembrane context (Gurezka et al., J. Biol. Chem. 274:9265-9270; Zhou et al. Nat. Str. Biol. 7:154-160). Arndt et al. (J. Mol. Biol. 295:627-639) describe an elegant approach to identification of novel heterodimeric coiled coil pairs via an in vivo protein fragment complementation assay.

In one embodiment, a dimerization domain containing a leucine zipper is modified by inserting a detection domain at one end of the leucine zipper motif. (Insertions within the leucine zipper are disfavored, as they are likely to seriously disrupt formation of the leucine zipper and/or the coiled coil interactions.) If the detection domain is a ligand binding domain, binding of the ligand may sterically interfere with dimerization. Alternatively, ligand binding may induce an allosteric change in the protein that, depending on the choice of ligand binding domain and its placement, promotes or hinders dimerization.

Stimulus-responsive dimerization domains mediating heterodimerization are particularly useful in some embodiments. For example, the mammalian proteins Fos and Jun each contain a leucine zipper causing the proteins to heterodimerize with each other: formation of Fos-Jun heterodimers are energetically preferred over Fos-Fos or Jun-Jun homodimers. In one embodiment, an engineered heterodimeric transcription factor includes a lambda repressor DNA binding domain fused to the Fos leucine zipper and a Cro repressor DNA binding domain fused to the Jun leucine zipper; at least one of the leucine zippers, and preferably both leucine zippers, is (are) rendered stimulus-responsive by addition of an appropriate detection domain. The resulting engineered chimeric protein recognizes a novel, hybrid DNA sequence reflecting the combined DNA binding specificity of the two subunits, and activity of the engineered chimeric protein is stimulus-dependent.

AraC

The dimerization domain of araC includes eight antiparallel strands of a beta sheet followed by a long linker (Soisson et al., Science 276:421-425). The long linker is followed by a ninth beta strand and 2 alpha helices such that the alpha helices pack to one side of the beta barrel. Thus at the dimer interface there are two sets of coiled coil interactions. Candidate positions for placement of a detection domain include, for example, the loop between the two alpha helices of each coiled coil; the loop between strands 2 and 3; and the loop between strands 7 and 8. These loops are not believed to be part of the dimerization interface and are thus more likely to tolerate insertion of a heterologous peptide.

Lambda Repressor

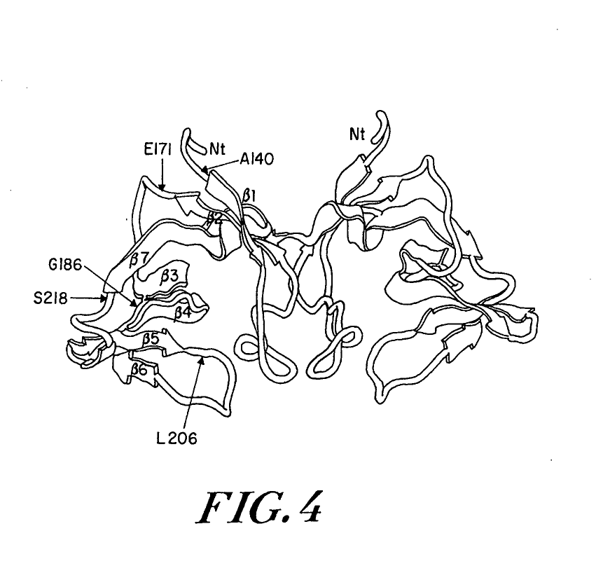

Based on the known structure of lambda repressor, there are several positions at which a short epitope of inserted sequence would not be expected to interfere with the dimerization of the repressor. These positions include, for example, insertions at amino acids 140, 171, 186, 206 and 218. The three-dimensional structure of the dimerization domain is shown in FIG. 4, with arrows pointing to the positions of interest. Insertions at these positions are likely to be tolerated since they are not in the beta sheets (which are integral to the structure) and they are not at sites already known through mutational analysis to be critical to function. Accordingly, these are good candidate positions for attaching or inserting a detection domain.

Lambda repressor can also be engineered by altering the linker (amino acids 92-132) connecting its DNA binding and dimerization domains. Much of the linker is dispensable: DNA binding and dimerization activities of the protein are retained even upon deletion of amino acids 93-129 (see Astronoff et al., Proc. Natl. Acad. Sci. USA 92:8110-4). If the linker is largely dispensable, it should be amenable to significant reengineering without unduly interfering with the protein's activity.

In one embodiment, a protein that can be induced to adopt a gross architectural change can be incorporated in the place of the flexible linker region. A derivative of maltose binding protein (MBP), a periplasmic E. coli protein, can replace or be inserted into the lambda repressor linker. The resulting protein should dimerize poorly: the dimerization domains would be out of position, and the extensive interactions between the first loop, the seventh beta strand and the carboxy-terminal helix of the dimerization domain of each monomer would be disrupted. Upon ligand binding, however, the domains of MBP move with respect to each other, inducing an eight degree twist and a thirty-five degree bend compared to the structure in the absence of ligand binding (see Sharff et al., Biochemistry 31(44): 10657-63). The conformational change is used to realign the dimerization domains, permitting dimerization to proceed. Thus, the engineered chimeric protein does not dimerize in the absence of ligand, but does dimerize upon ligand binding.

Importantly, MBP is susceptible to significant engineering. For example, artificial MBP derivatives with different ligand-binding specificities (e.g. binding zinc instead of maltose) undergo the same conformational change upon ligand binding (see Marvin et al., Proc. Natl. Acad. Sci. USA 98:4955-4960). Accordingly, in a preferred embodiment, MBP is engineered to contain a ligand binding peptide of the invention to render the protein responsive to a preselected ligand, and the engineered MBP protein is placed between the DNA binding and dimerization domains of lambda repressor, thereby to render dimerization of the engineered chimeric protein responsive to the ligand.

E2. Cooperativity

Proteins can also be regulated at the level of cooperative protein-protein interactions. For example, lambda repressor binds to DNA as a dimeric protein as described above. Lambda repressor also binds to DNA cooperatively if the DNA has two binding sites for lambda repressor. The cooperative binding occurs because a pair of lambda repressor dimers interact with each other while bound to the DNA, stabilizing the binding of each dimer to the DNA. Several papers have identified the amino acids that are required for the repressor to cooperatively interact and bind to DNA (Beckett et al., (1993) Biochemistry 32:9073-9079; Benson et al., (1994) Mol. Microbiol. 11:567-579; Burz et al., (1994) Biochemistry 33:8406-8416; Whipple et al., (1994) Genes Dev. 8:1212-1223; Whipple et al., (1998) Genes Dev. 12:2791-2802). As discussed above, a number of locations in the dimerization domain are good candidate locations for inserting a detection domain. Some of the proposed insertion sites, such as those at amino acids 186 and 206, are near the protein-protein interface at which two dimers interact to bind DNA cooperatively. An appropriately selected detection domain at one of these positions may have significant effects on cooperativity (as well as on dimerization). By increasing or decreasing cooperativity, the effective affinity of the dimers for the DNA (and, therefore, their effect on transcription) is modulated.

Cooperative binding of DNA binding proteins to a plurality of sites in a promoter usually requires that the spacing between the sites not exceed some maximum distance. Studies on the lambda repressor and AraC have shown that the maximum distance for cooperativity is reduced in proteins with reduced linker sizes (Astromoff, et al., Proc. Natl. Acad. Sci. USA 92:8110-8114; Eustance et al., J. Mol. Biol. 242:330-338). By replacing a portion of the linker with a ligand binding domain that adopts a more compact conformation upon ligand binding, it should be possible to mimic the effects of reduced linker length on cooperativity by simply adding ligand. Indeed, it has been suggested that arabinose may regulate AraC cooperativity by a similar mechanism (Carra, et al., EMBO J. 12:35-44.

E3. Transmembrane Proteins

Engineered ligand-responsive transmembrane proteins are particularly useful in sensing extracellular ligands. Cells contain many natural transmembrane proteins that monitor the environment for the presence of absence of particular analytes. Like a natural protein, an engineered ligand-responsive transmembrane chimeric protein includes an extracellular ligand binding domain, a transmembrane domain, and an intracellular domain that transduces the binding event into signaling events leading, for example, to the regulation of transcription of a target gene. Nevertheless, none of the domains of the engineered chimeric protein need be a naturally occurring domain; for example, the transmembrane domain may be created de novo using computational methods (reviewed in Ubarretxena-Belandia et al., Curr. Opin. Str. Biology 11:370-375). Generally, the transmembrane protein is engineered such that protein dimerization is responsive to a ligand; dimerization is an important step in activation of many natural transmembrane receptors. Alternatively, the transmembrane protein is engineered to adopt a conformational change upon ligand binding. The conformational change is communicated through the transmembrane domain to the intracellular domain where it affects the interaction of the intracellular domain with target biomolecules.

For example, the bacterial toxR protein includes an extracellular domain, a transmembrane domain, and an intracellular domain that binds to DNA and regulates transcription of a target gene. In some systems, the activity of toxR is believed to be modulated by dimerization of the protein, promoting its cooperative interaction with tandem DNA binding sites in the promoter of a target gene. As discussed above, there are numerous ways to engineer a ligand-responsive dimerization domain. Replacing the natural toxR extracellular domain with a ligand-responsive dimerization domain (or, perhaps, inserting a ligand-responsive dimerization domain into the natural extracellular domain) permits the regulation of a toxR responsive gene by the presence or absence of a preselected ligand.

In eukaryotes, signal transduction pathways connecting transmembrane proteins and intracellular events, such as the JAK/STAT, PDGF, and EGF signal transduction pathways, are well characterized (see Bromberg et al., Oncogene 2000 May 15; 19(21):2468-73; ten Dijke et al., Trends Biochem. Sci. 2000 February; 25(2):64-70; Heldin et al., Physiol. Rev. 1999 October; 79(4):1283-316; Beyersmann EXS 2000; 89:11-28; Carter et al., J. Biol. Chem. 1998 Dec. 25; 273(52):35000-7). Extracellular signals from inducers are transformed into transcriptional and physiologic effects within the cell. Chimeric molecules comprising the receptor transmembrane domain and an engineered extracellular domain may be used to drive regulated transcription from reporter constructs. For example, the epitope selected above may be appended to receptor. When the chimera is expressed on a cell surface, it would be expected to bind to a molecule of the type that the epitope directs and then send a signal into the cell; which would respond to the stimulus by turning on a reporter allele. This reporter allele may be able to be sensed directly, or the cell's phenotype may be altered to aid in detection of the receptor-ligand binding event.

Epidermal Growth Factor Receptor (EGFR) is an example of the growth factor receptor tyrosine kinase family that is anchored in the cell membrane by a single transmembrane domain (reviewed in Beyersmann EXS 89:11-28). The N-terminal extracellular domain is involved in binding not only its cognate ligand, EGF, but also heparin binding EGF-like growth factor, transforming growth factor alpha, amphiregulin, betacellulin and epiregulin (Gschwind et al., Oncogene 20:1594-1600). The intracellular part of the receptor contains a tyrosine kinase that is normally activated by ligand binding. Ligand binding is generally believed to promote dimerization of the receptor, promoting activation, although it has been suggested that activation may instead result from a conformational change communicated to the intracellular domain. EGFR tolerates at least a nine amino acid insertion between the extracellular and transmembrane domains; EGF binding and EGF-responsive tyrosine kinase activity are retained (Moriki et al., J. Mol. Biol. 311:1011-1026).

In one embodiment, an engineered ligand-responsive transmembrane chimeric protein is created by replacing the extracellular domain of EGFR with an engineered ligand binding domain of the invention. In another embodiment, an engineered ligand binding domain is introduced into the existing EGFR binding domain. The ligand binding domain preferably includes a ligand binding peptide that is no more than about fifty amino acids and is preferably engineered using information from a recombinant display technique. Ligand binding induces intracellular signaling by promoting receptor dimerization or by inducing a conformational change that is transduced to the intracellular domain. In a preferred embodiment, a ligand-responsive dimerization domain (as described above) is appended to the extracellular end of the transmembrane domain to promote ligand-dependent dimerization of the construct. Ligand-dependent activity is tested using any EGF-responsive promoter construct, such as a construct in which expression of a luciferase gene is controlled by the c-Fos gene enhancer v-sis inducible element (Souriau et al., NAR 25:1585-1590). Testing is preferably performed in a cell line that does not express EGFR, such as the B82L mouse fibroblast cell line (Cunnick et al., J. Biol. Chem. 273:14468-14475).

V. Additional Domains

Although an engineered stimulus-responsive protein of the invention includes at least an interaction domain and a detection domain, the engineered chimeric protein may advantageously include additional domains. For example, the engineered chimeric protein may include a domain that targets the protein to a particular location in the cell, such as the plasma membrane, the nucleus, or a vesicle. A domain that affects the degradation rate of the protein, such as a domain targeting the protein for ubiquitination, is useful to facilitate regulation of the steady-state levels of the protein.

If the engineered stimulus-responsive protein is a DNA binding protein, it is often useful to include a transcriptional activation or repression domain to facilitate transcriptional regulation by the stimulus-responsive protein. Such additional domains are not always required. For example, lambda repressor represses transcription at some promoters simply by binding to DNA and blocking access of RNA polymerase to the gene—no additional domain is required. At other promoters, lambda repressor activates transcription: amino acids in the DNA binding domain of lambda repressor are positioned to contact RNA polymerase, facilitating contact of the RNA polymerase with the gene. Nevertheless, addition of heterologous transcriptional activation or repression domains generally renders the resulting engineered chimeric protein more versatile. For example, fusing a eukaryotic transcriptional activation or repression domain to a prokaryotic DNA binding domain allows the engineered chimeric protein to regulate eukaryotic transcription (see, for example, U.S. Pat. Nos. 5,464,758 and 5,989,910). Thus, by fusing a modular transcriptional activation or repression domain to an engineered stimulus-responsive chimeric protein, the range of cells in which the engineered stimulus-responsive chimeric protein is effective is greatly expanded.

VI. Testing the Constructs

Once a proposed engineered chimeric protein has been designed with detection and interaction domains, the construct is tested to determine whether the stimulus modulates its activity. Preferably, many related constructs are tested at the same time, in which several potential detection domains are tested at each of several positions in the interaction domain. This not only facilitates the identification of those engineered chimeric proteins that are indeed modulated by a chosen stimulus (e.g. that bind a target biomolecule only in the presence of the stimulus, or only in the absence of the stimulus), but facilitates the identification of larger numbers of these engineered stimulus-responsive chimeric proteins, which can then be further characterized based on stimulus sensitivity and specificity, for example.

A. Synthesizing the Constructs

Methods for synthesizing proteins are well known. Although chemical synthesis of a protein may be possible for very small proteins, protein synthesis using the biological translational machinery is widely preferred. As a first step, a nucleic acid encoding the engineered chimeric protein is generated using standard molecular biology techniques such as PCR and chemical oligonucleotide synthesis. Using the known genetic code, any of a multiplicity of nucleic acids can be generated that encode a desired engineered chimeric protein. The nucleic acid is then generally cloned into an expression vector that places the nucleic acid encoding the engineered chimeric protein next to an active or inducible promoter. The expression vector is introduced into a cell, where the nucleic acid is transcribed and the protein is synthesized, or is transcribed and translated using in vitro systems known in the art. The expression vector may be introduced into cells by exposing the cells to the vector under conditions permitting uptake of the vector, by calcium chloride, calcium phosphate transfection, by treating the cells with a virus that injects the vector into the cells, or by other means known in the art. The protein is then optionally purified from the cell or from the in vitro translation system. For example, the engineered chimeric protein may be designed to incorporate a cluster of histidine amino acids (e.g. a cluster of six) to facilitate purification using a substrate comprising nickel ions capable of selectively binding the histidine cluster.

B. Exposing the Constructs to a Stimulus

In Vitro Assays

The preferred environment for testing an engineered chimeric protein depends on the nature of the engineered chimeric protein. For example, if the interaction to be regulated involves binding a target protein and phosphorylating it, testing may be done in vitro. The engineered chimeric protein is provided in a solution with the target protein and a phosphate source (e.g. ATP) in the presence or absence of the stimulus. Preferably, one or more other (e.g. unrelated) stimuli are also tested to determine the specificity of any observed responsiveness to the stimulus. Thus, for example, if an engineered chimeric protein is designed to respond to estrogen, it would be useful to test for activity in the absence of any ligand, in the presence of estrogen, and in the presence of other steroids such as progesterone and testosterone. In this context, one preferred construct would be active in the presence of estrogen but inactive in its absence, even in the presence of other steroids. Another preferred construct would be inactive in the presence of estrogen, but active under each of the other conditions. If a stimulus affects phosphorylation of a target protein, the phosphorylation event can be detected by any of a variety of methods including detecting a change in the mass or charge of the target protein (e.g. by mass spectrometry or electrophoretic mobility assays), detecting a change in the affinity of the target protein for an antibody that specifically binds the phosphorylated form of the protein, or by detecting incorporation of a radiolabeled phosphate group. These assays are routine in the art and can be performed in quantity to test a large number of potential engineered stimulus-responsive chimeric proteins.

Many other in vitro tests for detecting a binding interaction are known. For example, a binding event leads to a detectable increase in the mass of the complex, detectable by the changes in the behavior of the complex in an electrophoretic mobility assay, chromatographic assay, or surface plasmon resonance assay, among others. These assays can be performed in the presence and absence of a stimulus, and a difference in the size of the complex under the different conditions is detectable.

In Vivo Assays

In some instances it may be desirable to test a construct inside a living cell. If, for example, the engineered chimeric protein is a DNA binding protein that regulates transcription, it may be preferable to assay the effects of the protein on transcription rather than merely testing its ability to bind to DNA. Testing the engineered chimeric protein for its effects on transcription may be possible using an in vitro transcription system, but in vivo testing is generally preferable for this purpose.

If the engineered chimeric proteins are to be tested in a cell, they are preferably synthesized within that cell by administering an appropriate nucleic acid as described above. The cell preferably includes a reporter gene whose activity is to be regulated by an engineered stimulus-responsive chimeric protein. Regulation may be direct (e.g. if the engineered stimulus-responsive chimeric protein that binds to DNA) or indirect (e.g. if the engineered stimulus-responsive chimeric protein is a transmembrane protein that initiates a signaling cascade leading to regulation of the reporter gene).

A reporter gene directly or indirectly causes an effect detectable from outside the cell. Reporter genes are well known in the art and include, for example, glucuronidase, bacterial chloramphenicol acetyl transferase (CAT), beta-galactosidase (B-gal), various bacterial luciferase genes encoded by Vibrio harveyi, Vibrio fischeri, and Xenorhabdus luminescens, the firefly luciferase gene FFlux, green fluorescent protein, and the like. Reporter genes also include selectable markers such as antibiotic resistance genes and auxotrophic markers that modulate the viability of a cell. Alternatively, expression of a reporter gene may induce secretion of a growth factor such as FGF, EGF, PDGF, cytokines, and the like, which regulate proliferation, migration, and/or morphogenesis of cells to which they are exposed. In an alternate embodiment, a reporter gene induces production and/or secretion of a cell death signaling peptides, including but not limited to Fas ligand, Tumor necrosis factor (TNF) and the like, regulating the apoptosis of cells to which they are exposed.

When testing of engineered chimeric proteins is performed in a cell, if the stimulus is a ligand, the ligand must be able to reach the engineered chimeric protein. If the engineered chimeric protein is a transmembrane protein and the ligand binding domain is extracellular, it is sufficient to provide the ligand in a solution in contact with the cell. If, however, the engineered chimeric protein is intracellular, providing the ligand extracellularly is insufficient unless the cell is permeable to the ligand. For example, the ligand may be hydrophobic and able to pass directly through the cell membrane, or the ligand may be transported actively or passively by one or more transport proteins in the membrane.

Instead of being added to a cell extracellularly, the ligand may be synthesized within the cell. For example, if the ligand is a protein, a nucleic acid encoding the ligand may be introduced into the cell. One cell or population of cells is engineered to express the ligand, and another cell or population of cells is not. If the engineered chimeric protein is ligand-responsive, expression of the reporter gene in the two cells or cell populations will be differ. More preferably, expression levels of the ligand in the cell are regulatable by events external to the cell. For example, the ligand may be the mammalian p53 protein, whose steady-state protein levels in a cell are inducible by exposing the cell to ultraviolet radiation, or may be the phosphorylated form of a protein that is phosphorylated in response to EGF signaling. By treating the cell with an appropriate stimulus (e.g. UV radiation or an antibody that crosslinks the EGF receptor), the ligand is induced within the cell. The cell can then be tested for the activity of the engineered chimeric protein under induced and uninduced conditions by monitoring the effect of the reporter gene. Similarly, if expression of the ligand is regulated by an inducible promoter (e.g. a lactose-inducible promoter), expression of the ligand may be induced, permitting comparison of reporter gene activity in the induced and uninduced states.

Selections and Screens

Selections and screens for cells with a desired function are common in genetics and molecular biology and are effective in identifying engineered stimulus-responsive chimeric proteins among a library of candidate engineered chimeric proteins. In a selection, cells that lack the desired function are killed or fail to reproduce; only cells that have the desired function survive and proliferate. For example, in Saccharomyces cerevisiae, cells that lack a functional URA3 gene are unable to grow unless provided with an external source of uracil. Transcription of the URA3 gene can be made dependent on the activity of an engineered stimulus-responsive chimeric protein by, for example, placing the URA3 gene under the control of a promoter responsive to the engineered chimeric protein of interest. If a preselected stimulus modulates the binding of the engineered chimeric protein to the URA3 promoter, URA3 expression will be regulated by the presence or absence of the stimulus. Thus, if the engineered chimeric protein is a transcriptional activator and binds to DNA only in the absence of the stimulus, URA3 will be expressed only in the absence of the stimulus. (The opposite is true if the fusion protein is a transcriptional repressor and it binds to DNA only in the absence of the stimulus.) If the chimeric transcriptional activator binds to DNA only in the presence of the stimulus, URA3 will be expressed only in the presence of the stimulus. (The opposite is true if the fusion protein is a repressor and it binds to DNA only in the presence of stimulus.)

Using this system (or similar systems), selection strategies can be designed to select engineered chimeric proteins that respond to a preselected stimulus by turning on or off the expression of a selectable marker. For example, a library of nucleic acids encoding candidate engineered chimeric proteins may be introduced into yeast cells in which URA3 expression depends upon the binding of the engineered chimeric protein to a target biomolecule (using, for example, the methods and strains disclosed in U.S. Pat. No. 5,955,280 to Vidal et al.). The cells are then grown in the absence of uracil. Suppose the engineered chimeric protein is a transcriptional activator, and the desired engineered ligand-responsive chimeric protein will be active only in the absence of the stimulus. If neither ligand nor external uracil is provided to the cells, only those cells bearing engineered chimeric proteins that are active in the absence of the stimulus survive. In contrast, when treated with 5-fluoro-orotic acid (5-FOA), cells expressing URA3 are selectively killed. Accordingly, if the cells that survived the first selection are then exposed to the stimulus and 5-FOA (and provided with an external source of uracil), only those cells that have ceased expressing URA3 survive—the cells containing engineered chimeric proteins that are selectively inactivated in the presence of the stimulus. This is summarized in the following table:

Selection for transcriptional activator
active only in the absence of ligand.

| | Selection for | Selection against |
|---|---|---|
| | URA3 expression | URA3 expression |
| Conditions: | No uracil, no | Uracil and stimulus |
| | stimulus, no | and 5-FOA |
| | 5-FOA | |
| Engineered chimeric | No URA3 expression: | No URA3 expression |
| protein inactive | Killed by lack | Survival. |
| regardless of | of uracil | |
| stimulus | | |
| Engineered chimeric | URA3 expressed. | URA3 expressed: |
| protein active | Survival. | Killed by 5-FOA. |
| regardless of | | |
| stimulus | | |
| Engineered chimeric | No URA3 expression: | URA3 expressed. |
| protein active only | Killed by lack | Killed by 5-FOA. |
| in the presence | of uracil. | |
| of stimulus | | |
| Engineered chimeric | URA3 expressed. | No URA3 expressed. |
| protein active only | Survival. | Survival. |
| in the absence | | |
| of stimulus | | |

Thus, the only cells that survive the above selection strategy are those transformed with a nucleic acid encoding an engineered stimulus-responsive chimeric protein that is active in the absence of the stimulus but not in its presence. The same selection strategy can be used to select transcriptional repressors active only in the presence of the stimulus. If the selection is changed by adding the stimulus in the selection for URA3 expression and not in the selection against URA3 expression, the strategy will select transcriptional activators active only in the presence of the stimulus or transcriptional repressors active only in the absence of the stimulus. The surviving cells are allowed to multiply and the nucleic acid encoding the engineered chimeric protein is isolated using standard techniques. Once characterized, the engineered stimulus-responsive chimeric protein is also useful in other organisms and, in some embodiments, in vitro.

Screening strategies are very similar to selection strategies, except that expression of the reporter gene is evidenced by an effect other than a change in viability or reproduction. For example, a cell may change color or fluoresce in response to the reporter gene, which can be detected, for example, by a fluorescence-activated cell sorter (FACS) scanner. Selection and screening strategies can rapidly analyze up to tens of thousands of members of a library in a single experiment. Accordingly, many detection domains can be analyzed at each of many positions of an interaction domain and tested for proper function. In one embodiment, the detection domains are inserted at random positions in an interaction domain using combinatorial methods such as DNA shuffling or incremental truncation libraries (see, for example, PCT publication WO00/72013) to generate a library of candidate engineered chimeric proteins. Most members of such a random library will not encode functional engineered chimeric proteins. Those members, however, are selected or screened out using methods like those described above. Only the cells with nucleic acids encoding engineered stimulus-responsive chimeric proteins will pass through the selection or screen. Accordingly, these techniques provide a powerful technique for identifying engineered stimulus-responsive chimeric proteins even in the absence of preexisting structural or functional information about the interaction domain.

VII. Sensor Cells

A sensor cell can be constructed by expressing an engineered stimulus-responsive chimeric protein in a cell containing a reporter gene whose expression is regulated by the activity of the engineered stimulus-responsive chimeric protein. A sensor may also be engineered to include other components, such as engineered receptors, signaling molecules, actuators, etc. Any cell amenable to molecular biology techniques can be used, including, for example, bacterial cells, yeast cells, insect cells, fish cells, amphibian cells, bird cells, and mammalian cells (e.g. human cells). The cell can then be placed in a variety of environments to test for an event that triggers the engineered stimulus-responsive chimeric protein.

Contaminants, Fermentation Processes, and Etiologic Agents

A sensor cell can be used to detect the presence of a molecule in a contacting solution. The molecule may be the stimulus, in which case the detection domain of the engineered chimeric protein is preferably extracellular or the cell membrane is preferably permeable to the molecule. Alternatively, the molecule may indirectly induce presentation of the stimulus to the engineered chimeric protein, for example by inducing a signaling cascade regulating synthesis or degradation of the ligand.

The molecule to be detected may be a contaminant in a chemical process or product, a fermentation process, or in a food product, for example. Contaminants in chemical processes can indicate that a reaction is proceeding inefficiently; contaminants can also themselves disrupt a chemical process, slowing it and/or promoting unwanted side reactions. Efficient detection of contaminants can provide significant cost savings in large scale refining or chemical production by averting these inefficiencies. A sensor cell can detect these contaminants if the sensor cell is exposed to samples from the solution being processed. The expression of the reporter gene is modulated by the presence or absence of the contaminant. The effect of the expression of the reporter gene (e.g. fluorescence) is noted by an individual responsible for the process, who then takes action as appropriate.

Alternatively, the molecule may be an etiologic agent. For example, the military and civil protection authorities need a tool for rapidly detecting any of the many etiologic agents that may be used in biowarfare. A solution or suspension can be tested for the presence of an etiologic agent by contacting an appropriately engineered sensor cell with the solution and detecting the effect of the reporter gene.

Detecting and Treating Disease

The molecule may also be a disease marker, such as a molecule from a bacterium, virus, parasite, or a diseased cell, or a biomolecule such as a protein, nucleic acid or carbohydrate whose concentration or state tends to be different in healthy and unhealthy individuals. The sensor cell may be introduced into the body of a patient to directly or indirectly detect the presence of the disease, or may be exposed to a tissue or fluid sample from the patient. In a preferred embodiment, the sensor cell is introduced into the body using a capsule as described in U.S. Pat. No. 5,704,910, facilitating implantation and removal of the sensor cell.

In another preferred embodiment, the sensor cell is engineered to treat a disease. The sensor cell is implanted into a patient and designed to detect a locally abnormal state, such as a malignant, premalignant, or diseased cell, an abnormal protein plaque, or an etiologic agent. Upon detecting the abnormal state, the sensor cell responds by secreting a molecule that tends to counteract, neutralize, or eliminate the abnormal state.

Drug Discovery

Often, a drug that can regulate a biochemical pathway is a very effective pharmaceutical agent. For example, cancer is treatable by reducing cell growth, increasing cell apoptosis, or reducing angiogenesis. An engineered ligand-responsive chimeric protein can be designed to respond to an intracellular ligand whose levels reflect the activity of a biochemical pathway. A sensor cell containing such an engineered ligand-responsive chimeric protein is then an effective tool for screening drug candidates for their efficacy in regulating the pathway. If, after exposure of the sensor cell to a drug candidate, the expression of the reporter gene changes, the drug candidate presumably modulates the targeted biochemical pathway.

The sensor cell is also useful in screening for molecules with a desired biochemical activity. A library of candidate molecules is introduced into a population of sensor cells. Those cells containing molecules with the desired biochemical activity are identifiable based on the effects of the molecules on the biochemical pathway monitored by the engineered stimulus-responsive chimeric protein.

VIII. Cell-Based Logic

The engineered stimulus-responsive chimeric proteins and cells as described above are useful for many applications. One major application of these sensors and switches is in the realm of cell-based logic. Cell based logic may be described as the predictable programmatic action of a cellular or acellular system that will regulate biological or biochemical activity in response to a plurality of signals or to carry out complicated biological analysis in a manner analogous to electronic logic devices. By ganging layers of stimulus-responsive switches, robust logic circuits may be engineered. The desired generic logic devices that are expected to be duplicated in biological space include binary switches, NOR, OR, NOT, AND, and NAND gates, analog-to-digital converters, and digital-to-analog converters.

A. Binary Switches

In one preferred embodiment, target biomolecules of engineered stimulus-responsive chimeric proteins or other proteins are nucleic acids, such as protein binding sites in an operator or promoter.

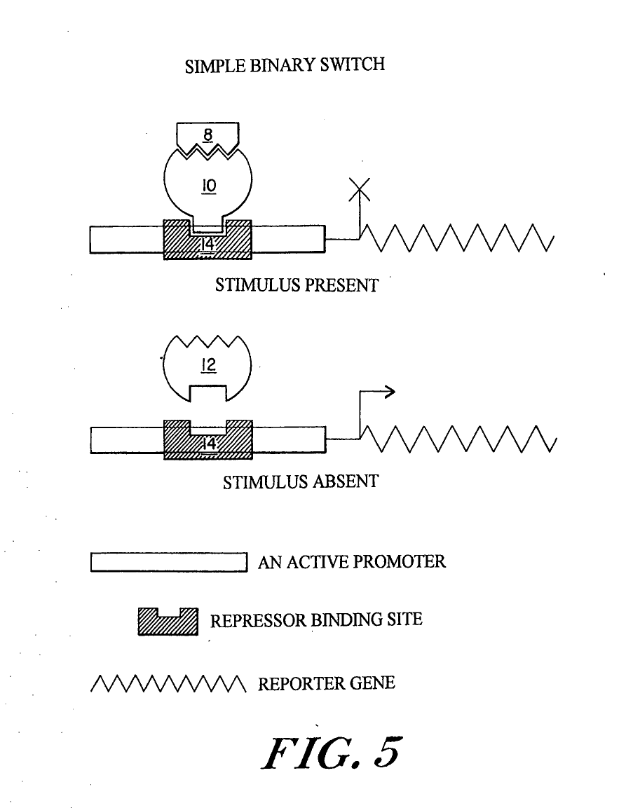

Transcription can be regulated as a binary switch having an active and an inactive state. (see, e.g., Biggar et al., EMBO J 20(12): 3167-3176 (2001); Becskei et al. EMBO J. 20(10): 2528-2535 (2001)). Bistable toggle switches and oscillatory networks have been constructed in Escherichia coli (see Gardner et al. Nature 403(6767):339-342 (2000); Elowitz et al. Nature 403:335-338 (2000)). One simple bistable switch includes an active promoter engineered with a repressor nucleic acid sequence that can be bound by an engineered stimulus-responsive chimeric protein. The interaction between the engineered chimeric protein and the binding site is regulated by the presence or absence of a stimulus. For example, in one embodiment illustrated in FIG. 5, when a ligand is present, ligand 8 switches engineered chimeric proteins from a free state 12 to a bound state 10. Proteins in bound state 10 associate with repressor site 14, switching off transcription. Conversely, in the absence of ligand 8, the engineered chimeric protein exists in free state 12 that fails to bind the repressor site 14 and the promoter is active.

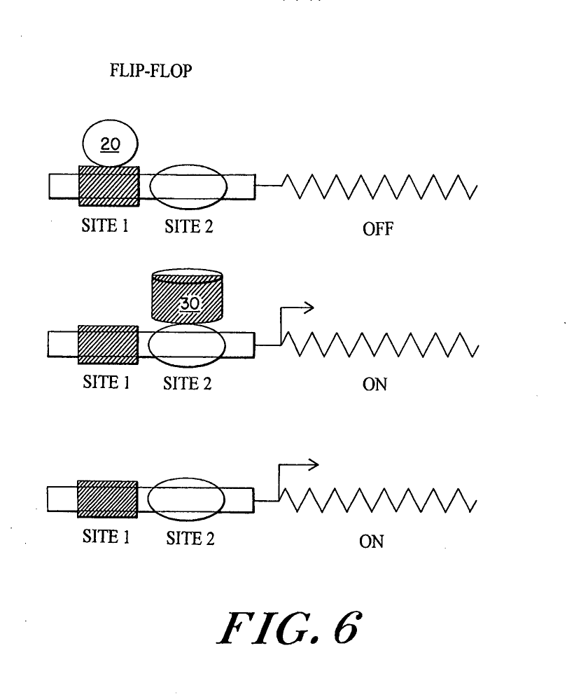

In another preferred embodiment, a binary transcriptional switch is designed to respond to two competing stimuli. For example, as depicted in FIG. 6, an active promoter can be engineered with two protein binding sites, one of which can be bound by a repressor 20, e.g., an engineered stimulus-responsive chimeric protein, and the other of which can be bound by an activator 30, e.g., a natural protein, an engineered protein, or an engineered stimulus-responsive chimeric protein. The two binding sites are situated close to each other so that when a first site is bound by its interacting protein the second site cannot be bound (e.g., due to steric hindrances). Conversely, when the second site is bound by its interacting protein, the first site cannot be bound. Therefore, the two sites can thus exist in two possible mutually exclusive states; either the first site bound or the second binding site bound.

If, for example, stimulus A for the chimeric repressor protein is present, the engineered chimeric protein binds the repressor binding site switching off transcription. If stimulus B (e.g., a developmental signal, a signal from another signaling pathway or an extracellular stimulus) is present to activate the activator, the activator binds to the activator binding site switching on transcription. If both stimuli are present, the chimeric repressor protein will oppose the effect of the activator and vice versa. The state of the transcription will be determined by the strength of the two regulatory sites (for example, if the repressing site is a higher affinity site, the chimeric repressor displaces the activator, turning off transcription; if the activating site has a higher affinity, the activator displaces the repressor, turning transcription on). If neither stimulus A nor stimulus B is present, neither protein binds its corresponding binding site. Since the promoter is active therefore, transcription is on. Such a device is also known as a molecular “flip-flop” that can be used to store information in a molecular binary computational or control system (see PCT publication WO 99/42929). The final readout of the molecular computational system is preferably the activity of a reporter gene that is operatively linked to the engineered promoter as described above.

A binary switch can also be designed as a logic gate to return a binary output signal that is a function of one or more inputs. The output and input signals can be described as having HIGH or LOW states. The input signals are carried (indicated) by engineered stimulus-responsive chimeric proteins, and/or other natural or engineered proteins that include an interaction domain that binds to a target biomolecule (e.g. a nucleic acid sequence). The output signal is preferably transcription of a reporter gene. The input signal states may be represented by the occupancy of one or more protein binding sites in the promoter of the reporter gene, the signal state being referred to as HIGH the site or sites are occupied and as LOW when unoccupied. The output signal state is HIGH when transcriptionally active and LOW when transcriptionally inactive.

Gates are well known to those of skill in the art. Basic gates include an AND gate, an OR gate, and an Inverter (the NOT function). Other gates include the NOR (NOT OR), the NAND (NOT AND), the exclusive OR (XOR), and so forth. A detailed description of gates can be found for example, in Horowitz and Hill (1990) The Art of Electronics, Cambridge University Press, Cambridge. Gates regulated by nucleic acid binding proteins are disclosed, for example, in WO 99/42929; the engineered stimulus-responsive proteins of the invention may be advantageously used to engineer logic gates and circuits using the methods and techniques described therein, and/or as described below.

B. NOR Gate

The output of a NOR gate is HIGH (transcriptionally active) only when both inputs are LOW (unbound). This can be expressed in a “truth table” as shown in Table 1. In the truth tables shown herein, input refers to the occupancy of a nucleic acid sequence that can be bound by a protein (protein binding site) within a promoter sequence, while output refers to transcriptional state of a reporter gene operatively linked to the promoter comprising inputs. The inputs are viewed as HIGH when bound by a protein (e.g., an engineered stimulus-responsive chimeric protein, a natural or engineered protein) and as LOW when they are not so bound. The output is HIGH when the transcription of the reporter gene is activated. Conversely the output is LOW when the transcription of the reporter gene is repressed. A “I” in the truth tables shown herein represents a HIGH state, while a zero represents a LOW state.

TABLE 1
The truth table of a NOR gate.

| Input 1 | Input 2 | Output |
|---|---|---|
| I₁ | I₂ | 0₁ |
| 0 | 0 | 1 |
| 0 | 1 | 0 |
| 1 | 0 | 0 |
| 1 | 1 | 0 |

As illustrated in Table 1, the NOR gate output is HIGH only when both inputs are low. If there are more than two inputs, the NOR gate output is HIGH only when all of the inputs are low. If any input is set HIGH, the output of the NOR gate is LOW.

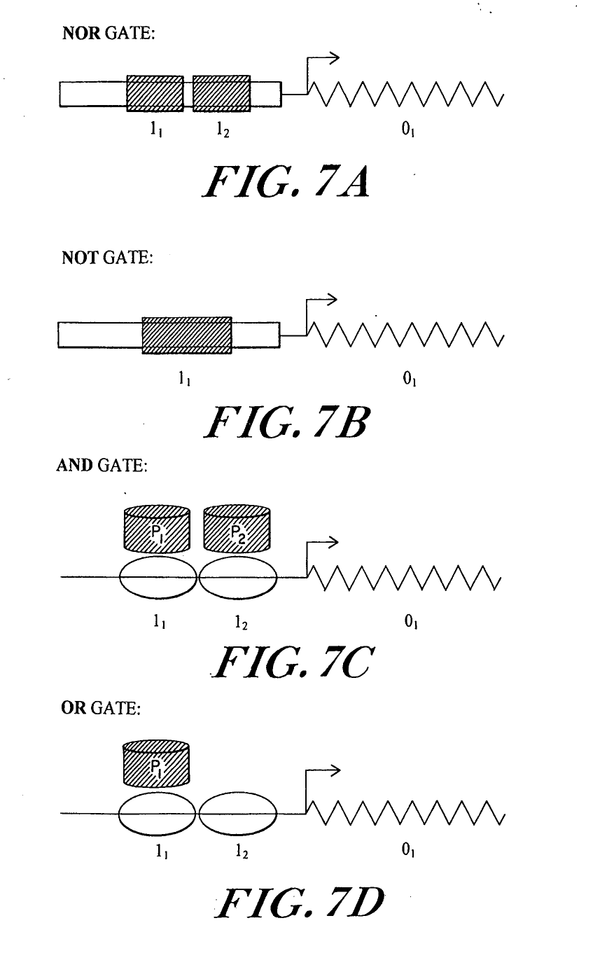

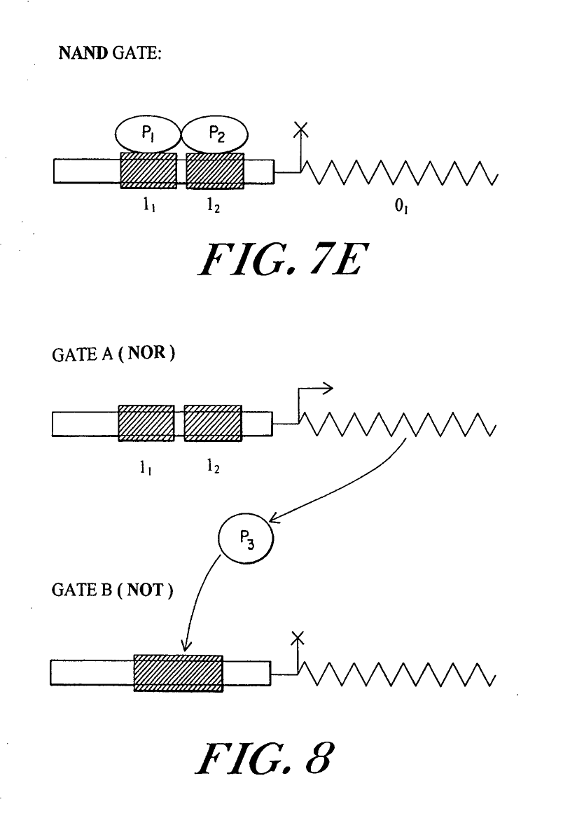

One example of a molecular NOR gate of this invention is illustrated in FIG. 7A. A preferred NOR gate includes an active promoter nucleic acid sequence having at least two repressor binding sites, designated I₁, and I₂. When either input site (I₁ or I₂) is bound by a repressor protein, the promoter is unable to initiate transcription of the reporter gene, designated as “output” (O₁). At least one of the input sites can be a binding site for an engineered stimulus-responsive chimeric protein.

Under these circumstances, the conditions of Table 1 are met. If either input protein binding site is bound with a protein, the transcription is repressed. The only condition when the output is HIGH (transcriptionally active) is when both inputs are LOW (unbound).

C. Inverter (Not) Function.

A second important combinatorial logic function is the inverter or NOT function. The NOT function returns the complement of a logic level. The NOT function is illustrated by the truth table of Table 2.

TABLE 2
Truth table of the NOT (inverter) function.

| Input 1 | Output |
|---|---|
| I₁ | 0₁ |
| 0 | 1 |
| 1 | 0 |

A NOT function returns a LOW signal state when the input is HIGH and a HIGH signal state when the input is LOW. An example of a NOT gate is illustrated in FIG. 7B. A preferred NOT gate includes an active promoter having a repressor binding site, designated as I₁. Binding of a repressor protein (e.g., an engineered stimulus-responsive chimeric protein, a natural or engineered protein) to an input (thereby setting the input HIGH) prevents transcription (thereby setting the output LOW).

D. AND Gate

The output of an AND gate is HIGH (transcriptionally active) only when both inputs are HIGH. This can be expressed in a “truth table” as shown in Table 3.

TABLE 3
The truth table of an AND gate.

| Input | Input | Output |
|---|---|---|
| I₁ | I₂ | O₁ |
| 0 | 0 | 0 |
| 0 | 1 | 0 |
| 1 | 0 | 0 |
| 1 | 1 | 1 |

One example of an AND gate of this invention is illustrated in FIG. 7C. A preferred AND gate includes an inactive promoter having at least two co-activator binding sites, designated I₁, and I₂. Neither co-activator alone is able to activate transcription: both co-activators are required (e.g., through cooperative interactions or dimerization) for activation of transcription. Under these circumstances, the conditions of Table 3 are met. If either input site is not bound by a co-activator protein, the output transcription is LOW. Only when both inputs are HIGH (bound) is the output HIGH.

E. OR gate

An OR gate is characterized by the truth table illustrated in Table 3.

TABLE 4
Truth table of an OR gate.

| Input 1 | Input 2 | Output |
|---|---|---|
| I₁ | I₂ | 0₁ |
| 0 | 0 | 0 |
| 0 | 1 | 1 |
| 1 | 0 | 1 |
| 1 | 1 | 1 |

Generally an OR gate produces a HIGH output (transcriptionally active) when any or all inputs are HIGH (binding sites are bound). An example of OR gate is illustrated in FIG. 7D. A preferred OR gate includes an inactive promoter having at least two activator binding sites. The activators are engineered stimulus-responsive chimeric proteins, and/or other engineered or natural protein. Either of the activators alone is sufficient to activate transcription. Under these circumstances, the conditions of Table 4 are met. If either input site is bound by a activator protein, the output transcription is HIGH.

F. NAND Gate

The output of a NAND (NOT AND) is shown in Table 5. The NAND gate is essentially an inverted AND gate. This gate produces a LOW output only when both inputs are set HIGH.

TABLE 5
The truth table of a NAND gate.

| Input 1 | Input 2 | Output |
|---|---|---|
| I₁ | I₂ | O₁ |
| 0 | 0 | 1 |
| 0 | 1 | 1 |
| 1 | 0 | 1 |
| 1 | 1 | 0 |

A NAND gate of this invention is illustrated in FIG. 7E. A preferred NAND gate includes an active promoter having at least two co-repressor binding sites, designated I₁ and I₂. Neither co-repressor alone is able to repress transcription: both co-repressors are required (e.g., through cooperative interactions or dimerization) for repression of transcription. Under these circumstances, the conditions of Table 5 are met. If either input site is not bound by a co-repressor protein, the output transcription is HIGH. The only condition when the output is LOW is when both inputs are HIGH (bound).

G. Combinations of Gates to Form Logic Circuits.

In the design of various gates and more elaborate molecular computing circuits it is often desirable to couple the output of one gate to the input of another gate. More particularly, the output of one gate acts as the input to one or more other gates.

For example, the output of a NOR gate can act as the input of a NOR gate to produce an OR gate (see PCT publication NO: WO 99/42929). In this case, the output (O₁) produced by two inputs (I₁ and I₂) is represented algebraically as:
O₁=OR(I₁,I₂)=NOT(NOR(I₁,I₂))

Coupling the output of one gate (or flip-flop) to the input of another gate (or flip-flop) can be accomplished by a number of means. For example, in a preferred embodiment, the output of one gate or “flip-flop” of this invention is transcription of a repressor or an activator that acts as an input into one or more other logic elements, i.e. other gates or “flip-flops” comprising nucleic acid sequences that can be bound by the repressor or the activator.

A simple example of coupling a NOR gate to a NOT gate is illustrated in FIG. 8. When both inputs are set LOW in the NOR gate A, it initiates transcription of a gene encodes a repressor protein P3 that, once expressed, can bind to the input binding sites of a NOT gate thereby setting the inputs HIGH, therefore the output transcription is set LOW. Similarly, the output of an AND gate can be coupled to an inverter, for example. More than two gates may be coupled and virtually any type of gate can be coupled to any other type of gate. Thus, various combinations of gates and/or “flip-flops” can be combined to produce complex computational logic and/or control circuits to process signals initiated by a plurality of stimuli. This can be accomplished by selecting and engineering appropriate input sites into appropriate promoters, and selecting or designing appropriate reporter genes encoding proteins having interaction domains that can bind to the preselected input sites. The expression of the reporter genes are regulated by other gates or “flip-flops”, which, in a preferred embodiment, include input sites that can be bound by engineered stimulus-responsive chimeric proteins, and/or natural or engineered proteins.

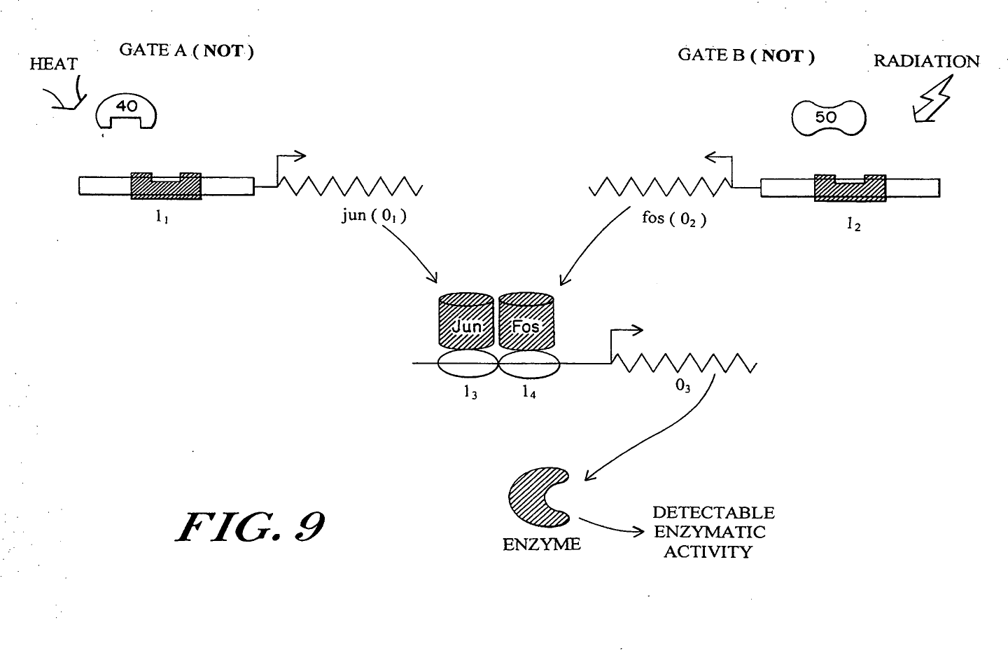

The logic circuits described above may be engineered in a single cell, e.g., a sensor cell, or in a population of cells to generate a multicellular circuit system in which the signaling output of one cell acts as input to another cell. In one embodiment, a sensor cell comprises a promoter AND gate that regulates the expression of a reporter gene encoding an enzyme, e.g., β-galactosidase. The preferred AND gate includes an inactive promoter containing two co-activator binding sites: one site can be bound by protein Jun, designated I₃ in FIG. 9, and the other site can be bound by protein Fos, designated I₄. The enzyme will not be expressed unless both Jun and Fos bind to their binding sites (both inputs HIGH). Within the same sensor cell, jun expression is under the control of a NOT gate A that includes an active promoter containing a repressor binding site I₁ that can be bound by an engineered temperature-responsive chimeric protein. An increase in temperature (or release of heat) induces a conformational change of first engineered chimeric protein 40 preventing it from binding to the input site I₁ in the NOT gate A; therefore, the transcription of jun is initiated and protein is synthesized. Within the same sensor cell, fos expression is under the control of another NOT gate B which includes an active promoter containing a repressor binding site I₂ that can be bound by an engineered chimeric protein responsive to electromagnetic irradiation. The presence of UV or other type of irradiation will prevent the binding of second engineered chimeric protein 50 to the binding site I₂ in the NOT gate B, therefore, the transcription of fos is initiated and protein is synthesized. As illustrated in FIG. 9, the enzyme expression is therefore under the control of both temperature and irradiation. Such a sensor cell can be used to monitor the heat and irradiation released during a nuclear reaction, a chemical reaction, environment pollution, and in other situations. The degree of heat and irradiation can be measured by measuring the activity of the reporter gene.

A cell based logic system can also be used to generate multistep, logically-contingent biological processes. These processes may be more complex than might occur through natural mutation, selection, or evolutionary processes because (1) the phase space required to discover such a process through natural means is too large (the program is too complex) or (2) the process employs a non-natural logical motif. For example, an artificial operon may be designed to control a metabolic process A->B->C such that the B->C step does not occur until the amount of product B reaches a certain threshold, perhaps by using an engineered stimulus-responsive chimeric protein that detects B to regulate synthesis of an enzyme to catalyze the B->C step. Similarly, the A->B step may be engineered to occur only when the amount of B is below a certain threshold, perhaps by using the engineered B-responsive chimeric protein to control synthesis or degradation of an enzyme catalyzing the A->B step. Such feedback regulation is common in natural operons and can be accomplished by programming biological logic circuits using engineered chimeric proteins. In other embodiments, an artificial operon may be designed to monitor multistep and/or quantitative biological processes including catalysis, synthesis, degradation and the like. These processes may be engineered in a cell or a population of cells. The cells may be programmed such that the output from one cell affects the output of another cell. Furthermore, population of such cells may be used to process large quantities of information using parallel processing techniques.

H. Analog Logic

Transcription can also be regulated in an “analog” fashion. In contrast to binary switch which turns a promoter either fully “on” or fully “off”, “analog” regulation allows a promoter response that achieves a range of activity between fully “on” to fully “off”. Analog regulation is also known as “graded” transcriptional regulation, and it is commonly used by eukaryotic cells. The advantage of analog logic is that the amount of signal readout is indicative of the amount of signal input. In a biological system, analog regulation may permit a cell or a multicellular system to fine-tune its response to allow a proportionate or differential response to a graded input stimulus.

A transcriptional analog promoter may, for example, be engineered by combining a weak promoter with any of a multitude of activator binding sites. Each activator may increase the transcriptional activity. The activators can be engineered stimulus-responsive chimeric proteins so that the transcription is regulated in proportion to the amount of a preselected input stimulus. The activators can also be other engineered proteins and natural proteins. An analog promoter can also be engineered by combining an active promoter with any of a multitude of repressor binding sites. Each repressor decreases the transcriptional activity. The repressors can be engineered stimulus-responsive chimeric proteins, other engineered proteins or natural proteins.

Analog regulation may be post-transcriptional. In one embodiment, regulatory sequences are engineered in a 3′ untranslated region of a reporter gene to regulate RNA stability, degradation or translation and the like. Proteins binding to these regulatory sequences may include engineered stimulus-responsive chimeric proteins rendering the regulation stimulus-responsive. In other embodiments, the regulatory sequences are regulated by binary switches, including NOR, AND, OR, NOT and NAND gates or “flip-flops”, so that the signal readout from a binary system has an analog dimension.

Digital-to-analog conversion may further achieved by directly coupling the gates and “flip-flops” to analog promoters. For example, in one embodiment, the reporter genes regulated by gates and flip-flops encode transcriptional regulators of an analog promoter. Therefore the outputs of binary systems act as input signals for an analog system. Similarly, analog-to-digital conversion may be achieved by designing reporter genes regulated by analog promoters to act as input signals for binary systems.

The combination of binary and analog logic system of the present invention allow a potent and flexible biological computing system that will essentially process any input signals to a desired level.

IX. Engineering of Artificial Signaling Systems

A. Engineered Receptors

Many receptor types are amenable to engineering into systems that interface with an engineered stimulus-responsive chimeric protein. For example, the tyrosine kinase, tyrosine/serine, dual specificity kinase type and Ras/MAPK camp/CREB, JAK/STAT and TGFβ receptors and second messenger systems are understood at the molecular level and are useful as scaffolds for engineered signaling cascades. Another among these receptor families that can be engineered is the G coupled protein receptors. These proteins can be designed to respond to specific engineered ligands. G coupled protein receptors (GCPR) are a diverse family of receptor molecules with varied functions whose activities have been extensively characterized (Hamm, H. E., D. Deretic, et al. (1988). “Site of G protein binding to rhodopsin mapped with synthetic peptides from the alpha subunit.” Science 241(4867): 832-5; Hamm, H. E. and A. Gilchrist (1996). “Heterotrimeric G proteins.” Curr Opin Cell Biol 8(2): 189-96; Hamm, H. E. (1998). “The many faces of G protein signaling.” J Biol Chem 273(2): 669-72; Gilchrist, A., M. Bunemann, et al. (1999). “A dominant-negative strategy for studying roles of G proteins in vivo.” J Biol Chem 274(10): 6610-6; Gether, U. (2000). “Uncovering molecular mechanisms involved in activation of G protein-coupled receptors.” Endocr Rev 21(1): 90-113; Gilchrist, A., A. Li, et al. (2000). “Use of peptides-on-plasmids combinatorial library to identify high-affinity peptides that bind rhodopsin.” Methods Enzymol 315: 388-404; Gouldson, P. R., C. Higgs, et al. (2000). “Dimerization and domain swapping in G-protein-coupled receptors. A computational study.” Neuropsychopharmacology 23(4 Suppl): S60-77). Engineered regulation of GCPR signaling has been demonstrated. Small molecules like norepinepherine and peptides have been used to regulate signaling from these receptors. Critical residues that bind to cognate ligands and those that bind to G interacting proteins have been described.

B. Signaling Molecules

Known signaling pathways can be harnessed both to regulate exposure of the engineered chimeric protein to the stimulus and to transmit effects of the engineered chimeric proteins of the invention. The targets of signaling may reside within the cell, may be extracellular, or may be other devices. For example, bioluminescence in the marine bacterium Vibrio fischeri is controlled by the excretion of an N-acyl homoserine lactone auto inducer, which interacts with a regulator, LuxR, and activates transcription of the lux operon at high cell density. The lux operon in V. fischeri is an example of an extracellular signaling (cell-cell) quorum-sensing mechanism. Each cell produces the product, which in turn produces a discrete amount of N-acyl homoserine lactone. In the presence of large numbers of the same organism, the N-acyl homoserine lactone concentration is elevated and the organism is induced to engage in transcription of the rest of the cascade. This system and small molecule may be used for the purpose of signaling the result of an interaction with one cell in a population with another through the use of an engineered sense/response construct within adjacent cells. Once signaled, the second cell may follow its own engineered program of sensing and then respond with another inducer, hormone, or light as in the case of BRET (Xu, Y., D. W. Piston, et al. (1999). “A bioluminescence resonance energy transfer (BRET) system: application to interacting circadian clock proteins.” Proc Natl Acad Sci USA 96(1): 151-6).

In yeast, Gβγ is the activator of a pheromone-stimulated MAP kinase pathway. It is known to bind to the N-terminal region of the scaffold protein Ste5 in yeast. Ste5 contains a homodimerization domain, which is required for P binding. Gβγ directs the oligomerization of this domain on Ste5. Chimeric constructs with the Gβγ domain fused with glutathione S-transferase activate the MAP kinase cascade. By co-opting and engineering GCPR and a protein containing this domain the directed activation of a specific MAP kinase and specific transcription events may be designed. Each of these elements and motifs are examples of what can be identified and engineered with this design process. Exemplary signaling molecules and cascades also include those regulated by PDGF, EGF, or ion channels.

C. Actuators

Similarly, actuators can be designed to respond to the activity of an engineered stimulus-responsive chimeric protein. Actuators useful in the practice of the present invention include any molecules or systems capable of altering the properties of a cell. For example, engineered actuators may include catalytic and anabolic enzymes, pumps and reporter constructs. Catalytic enzymes like RNA polymerase may be used to read an instruction from a DNA template in response to a specific chemical signal. Alternatively, an engineered calcium channel may be designed to report on the local concentration of Ca⁺⁺ inside the cell as a reporter of the activation state of the cell using “cameleon” proteins (Miyawaki, A., O. Griesbeck, et al. (1999). “Dynamic and quantitative Ca2+ measurements using improved cameleons.” Proc Natl Acad Sci USA 96(5): 2135-40) Cross receptor signaling may be accomplished by designing engineered SH2/3 adapter Grb2, SOS, MAPK, etc. interacting peptides, and kinases.

In one preferred embodiment, the actuator affects cell motility. For example, the elements of the bacterial chemotaxis system are described in sufficient detail to engineer chemotactic response of bacteria (Bray, D. and R. B. Bourret (1995). “Computer analysis of the binding reactions leading to a transmembrane receptor-linked multiprotein complex involved in bacterial chemotaxis.” Mol Biol Cell 6(10): 1367-80.; Shukla, D. and P. Matsumura (1995). “Mutations leading to altered CheA binding cluster on a face of Che Y.” J Biol Chem 270(41): 24414-9; Swanson, R. V., D. F. Lowry, et al. (1995). “Localized perturbations in CheY structure monitored by NMR identify a CheA binding interface.” Nat Struct Biol 2(10): 906-10; Eisenbach, M. (1996). “Control of bacterial chemotaxis.” Mol Microbiol 20(5): 903-10; Bass, R. B. and J. J. Falke (1998). “Detection of a conserved alpha-helix in the kinase-docking region of the aspartate receptor by cysteine and disulfide scanning.” J Biol Chem 273(39): 25006-14; Blat, Y., B. Gillespie, et al. (1998). “Regulation of phosphatase activity in bacterial chemotaxis.” J Mol Biol 284(4): 1191-9; Djordjevic, S. and A. M. Stock (1998). “Structural analysis of bacterial chemotaxis proteins: components of a dynamic signaling system.” J Struct Biol 124(2-3): 189-200; McEvoy, M. M., A. C. Hausrath, et al. (1998). “Two binding modes reveal flexibility in kinase/response regulator interactions in the bacterial chemotaxis pathway.” Proc Natl Acad Sci USA 95(13): 7333-8; Roychoudhury, S., S. E. Blondelle, et al. (1998). “Use of combinatorial library screening to identify inhibitors of a bacterial two-component signal transduction kinase.” Mol Divers 4(3): 173-82; Scharf, B. E., K. A. Fahrner, et al. (1998). “Control of direction of flagellar rotation in bacterial chemotaxis.” Proc Natl Acad Sci USA 95(1): 201-6; Welch, M., N. Chinardet, et al. (1998). “Structure of the CheY-binding domain of histidine kinase CheA in complex with CheY.” Nat Struct Biol 5(1): 25-9; Bilwes, A. M., L. A. Alex, et al. (1999). “Structure of CheA, a signal-transducing histidine kinase.” Cell 96(1): 131-41; Dutta, R., L. Qin, et al. (1999). “Histidine kinases: diversity of domain organization.” Mol Microbiol 34(4): 633-40.; Jasuja, R., Y. Lin, et al. (1999). “Response tuning in bacterial chemotaxis.” Proc Natl Acad Sci USA 96(20): 11346-51; Simon Shimizu, T., N. Le Novere, et al. (2000). “Molecular model of a lattice of signaling proteins involved in bacterial chemotaxis.” Nat Cell Biol 2(11): 792-796; Sola, M., E. Lopez-Hernandez, et al. (2000). “Towards understanding a molecular switch mechanism: thermodynamic and crystallographic studies of the signal transduction protein cheY” J Mol Biol 303(2): 213-25).

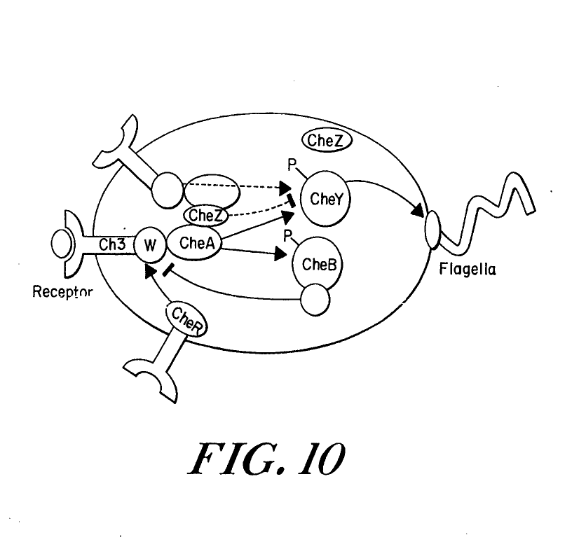

As shown in FIG. 10, the signaling events controlling bacterial chemotaxis begin with transmembrane receptor proteins binding chemoeffectors and, through an adapter protein CheW, controlling the activity of the histidine protein kinase CheA. The cytoplasmic domains of the receptors are methylated by methyltransferase CheR and demethylated by methylesterase CheB. Attractant binding decreases kinase activity, while receptor methylation increases kinase activity. CheA provides phosphoryl groups to CheY and CheB, producing active forms of these proteins. Phosphorylated CheB demethylates receptors, providing a feedback loop that contributes to adaptation. The response regulator, phosphorylated CheY, binds to the flagellar motor, inducing a clockwise flagellar rotation and a tumbling response. CheZ accelerates the dephosphorylation of CheY. The dashed lines indicate the possible routes for amplification of the excitation signal. The structure of these molecules are known and the interfaces of these proteins have been described to the molecular level (Bray, D. and R. B. Bourret (1995). “Computer analysis of the binding reactions leading to a transmembrane receptor-linked multiprotein complex involved in bacterial chemotaxis.” Mol Biol Cell 6(10): 1367-80.; Swanson, R. V., D. F. Lowry, et al. (1995). “Localized perturbations in CheY structure monitored by NMR identify a CheA binding interface.” Nat Struct Biol 2(10): 906-10; Zhu, X., C. D. Amsler, et al. (1996). “Tyrosine 106 of CheY plays an important role in chemotaxis signal transduction in Escherichia coli.” J Bacteriol 178(14): 4208-15; Abouhamad, W. N., D. Bray, et al. (1998). “Computer-aided resolution of an experimental paradox in bacterial chemotaxis.” J Bacteriol 180(15): 3757-64; Appleby, J. L. and R. B. Bourret (1998). “Proposed signal transduction role for conserved CheY residue Thr87, a member of the response regulator active-site quintet.” J Bacteriol 180(14): 3563-9; Da Re, S. S., D. Deville-Bonne, et al. (1999). “Kinetics of CheY phosphorylation by small molecule phosphodonors.” FEBS Lett 457(3): 323-6.) including the flagella. Bacteria use this system to migrate towards or away from chemicals present in a gradient. Cells directed to move in relation to a gradient can be used to pattern cell density on a surface, or in solution. The present invention can use this system to direct the migration of bacteria by altering the interfaces between and the primary sequences of these proteins to allow programmed control of locomotion.

D. Molecular Memory

A stimulus can be “remembered” by a cell by feeding the output of the engineered chimeric protein into a molecular memory device. Engineered, biological molecular memory elements may be devised using, for example, Cre/LoxP, invertase or kinase motifs, or genetic toggle switches. We propose a novel method of recording an event, or altering a program in a cell by the use of a modified Cre/LoxP, or invertase system. An event sensed by the cell can be transformed into the regulated expression of Cre recombinase or invertase. Alternatively, these enzymes may be delivered into the cell by other means like lipofection.

LoxP is a specific DNA sequence that is recognized by the bacteriophage P1 enzyme Cre recombinase. The LoxP site has been shown to contain a 34 bp motif, present in two copies. When the LoxP motifs are present in a DNA sequence contacted with the Cre recombinase enzyme, the Cre can excise a segment of the DNA in a predictable manner. Thus, an event sensed by the cell may be recorded in a “non-volatile” fashion by the excision of certain “reporter” DNA elements. The record of this excision may be read as a loss of function to a cell (auxotrophy), or as an orphan genetic element, which can be decoded by other biochemical means (e.g. PCR, or sequencing, etc). Similarly, invertase is an enzyme of bacterial origin, which allows the site-specific excision, inversion/silencing of DNA elements between specific sequences. This enzyme is also capable of being used in the design of a “non-volatile” memory as described above. The main difference lies in the fact that the invertase reaction retains the piece of DNA in between the two recombination sites and simply inverts its orientation. Readout mechanisms would be the same as in the Cre/LoxP system.

Alternatively, molecular memory may use specific phosphorylation of engineered target proteins. Phosphorylation of specific sequences in proteins has been described. Engineering these sequences in engineered chimeric proteins would allow the recording of an interaction of this protein with the specific kinase. The readout of this event may be the interaction, or inhibition of interaction of the phosphorylated protein with a reporter, or specific antibody. The peptide sequence -LRRASLG- (SEQ ID NO:

5) is the target sequence for protein kinase A (PKA), -RRREEETEEE- (SEQ ID NO: 6) is a substrate for casein kinase II, -EAIYAAPFAKKK- (SEQ ID NO: 7) is the substrate sequence for v-Abl Protein Tyrosine Kinase (PTK), etc. (Marshak, D. R. and Carroll, D. (1991) Methods Enzymol. 200, 134-156). These sequences may be included in a “recorder protein” which is constitutively expressed in a cell. When the “event” occurs, the cell would activate, or express the appropriate kinase activity. The protein would then be marked for the life of the protein with a sequence-specific phosphate group. In one embodiment, the reporter protein is preferably resistant to degradation and dephosphorylation to permit lasting “memory” of the phosphorylation event. Using this system in heterologous hosts like E. coli may allow the use of those kinases and phosphatases that might perturb the normal function of a eukaryotic cell.

E. Signal Initiators

Signal initiators can be adopted from naturally evolved inducible signaling pathways to render exposure of the engineered chimeric protein to the stimulus conditional upon some other biophysical stimulus. Many microorganisms, plants and mammals in nature have evolved different inducible adaptive systems to cope with the toxic effects of a wide range of stresses. For example, both cold shock and heat shock proteins help bacteria and other microorganisms cope with the variation of temperature (“Heat-shock proteins and stress tolerance in microorganisms.” Curr Opin Microbiol April; 4(2):166-71; Lindquist S. (2001). “Responses of Gram-negative bacteria to certain environmental stressors.” Cell Physiol Biochem; 10(5-6):303-6; Ramos J L et al (2000).) HSP72 expression is regulated in response to the osmotic stress and pH change in the solutions (“Heat shock proteins and the cellular response to osmotic stress. Mol Microbiol July; 29(2):397-407; Poolman B et al. (1998).) Plant stem cells response to gravitropic stimulation by a rapid and reversible change in elongation (“Cellular mechanisms underlying growth asymmetry during stem gravitropism.” Planta September; 203(Suppl 1):S130-5; Cosgrove D J. (1997)) Nature has also evolved different kind of systems to protect organisms from UV or electromagnetic radiation, such as SOS response system in bacteria, RAD superfamily proteins in yeast, and P53 in mammals (“A non-excision uvr-dependent DNA repair pathway of Escherichia coli (involvement of stress proteins).” J. Photochem Photobiol B. September; 45(2-3): 75-81; Sedliakova M. (1998). “Repair of UV-damaged DNA by mammalian cells and Saccharomyces cerevisiae.” Curr Opin Genet Dev April; 4(2):212-20; Aboussekhra A et al (1994). “Doing the right thing: feedback control and p53.” Curr Opin Cell Biol April; 5(2):214-8; Prives C. (1993).) These natural inducible systems can be adopted and engineered to transform the biophysical stimulus, such as temperature, osmolarity change, electromagnetic radiation and the like, to desired signals that can be presented to the downstream sensor cells to initiate signaling cascades.

IX. Multicellular Devices

In a multicellular logic circuit system, the logic output of one cell becomes a logical input for another cell. For example, one cell may secrete tetracycline in a lactose-dependent manner (program A), inducing a tetracycline-dependent program (program B) in a second cell. Each program is self-regulating and follows its preprogrammed algorithm. However, if program A feeds its output into program B, then the output of program B is contingent on program A. This is important if one desires the product of one cell to be dependent on another.

Many signaling mechanisms are available for use with the present invention. For example, in one embodiment, small molecules or peptides are synthesized and secreted by a first cell into an extracellular environment, and those small molecules and peptides subsequently enter a second cell and regulate gene expression in the second cell, perhaps by binding an engineered stimulus-responsive chimeric protein. In another embodiment, a peptide is synthesized and secreted by a first cell, and the peptide functions as a switch to initiate a signaling cascade in the sensor cell leading to a synthesis of a ligand inside a second cell; the ligand interacts with an engineered ligand-responsive chimeric protein to regulate transcription. Alternatively, the peptide activates a degradation process inside a second cell leading to the degradation of a ligand. The peptide may instead activate a signaling pathway leading to the relocation of a ligand inside the sensor cell so that the ligand becomes accessible to the transcriptional machinery. In another embodiment, a peptide is synthesized and expressed on an exterior surface of a first cell, and the extracellular part of the peptide interacts with a second cell, initiating a signaling cascade in the second cell.

The following examples are intended to illustrate certain preferred aspects of the invention and are not to be interpreted as limiting the scope of the invention in any way.

### EXAMPLE

### Design of a Taxol-Responsive Transcriptional Switch

Phage display experiments performed with biotinylated-taxol led to the identification of short peptides that exhibited homology to a 60 amino acid section of the Bcl2 protein (Rodi et al., J. Mol. Biol. 285:197-203). These 60 amino acids are predicted to be in a disordered loop of Bcl2. It has been demonstrated that taxol specifically bound to GST-Bcl2 with a Kd in the nanomolar range. The binding activity was further narrowed down to a 30 amino acid stretch (Rodi et al. J. Mol. Biol. 285, 197-203). Based on these studies, a 12-amino-acid-stretch from Bcl2 protein with extensive homology to the peptides identified by phage display was selected as the taxol binding domain (TBD).

Creation of Lambda Repressor Derivatives

It has been shown that the carboxy-terminal domain of lambda repressor (amino acids 133-236) can be substituted by dimerization domains from related repressors, as well as the unrelated leucine zipper dimerization domain. A functional chimeric repressor was created by fusing the DNA binding domain (DBD) and linker regions of lambda repressor (cI) with the 32 amino acid leucine zipper motif from the S. cerevisiae transcription factor, GCN4 (Hu et al., Science 250:1400): this chimeric cI derivative is referred to as cI-bZIP.

Specifically, oligonucleotides S5 (SEQ ID NO: 8) and S6 (SEQ ID NO: 9) were used to amplify the leucine zipper motif from S. cerevisiae GCN4. Oligonucleotide S5 contained an additional isoleucine at the 5′ end such that ligation of the PCR product into EcoRV cut pETBlue1 would regenerate the EcoRV site. Digestion of this plasmid by EcoRV followed by ligation to a blunt end PCR product corresponding to amino acids 1-132 of cI (generated by S1 (SEQ ID NO: 10) and S7 (SEQ ID NO: 11)) generated the chimeric repressor cI-bZIP (SEQ ID NO: 12).

Oligonucleotides Used:

| | |
|---|---|
| Oligo | |
| name | Sequence |

| | | |
|---|---|---|
| S1 | 5′ ATGAGCACAAAAAAGAAACCATTAAC | SEQ ID NO: 10 |
| | 3′ | |
| S2 | 5′ TTACAACGCCCGGGTCAGCCAAACGTGT | SEQ ID NO: 13 |
| | CTTCAGG 3′ | |
| S5 | 5′ ATCGCGCACATGAAACAACTTGAAGAC | SEQ ID NO: 8 |
| | 3′ | |
| S6 | 5′ TCAGCGTTCGCCAACTAATTTC 3′ | SEQ ID NO: 9 |
| S7 | 5′ GCTTACCCAGCGCTCCGC 3′ | SEQ ID NO: 11 |
| S8 | 5′ ATGGGCATTTTCTCGAGTCAGCCGGGCC | SEQ ID NO: 16 |
| | ATACCCCGCATCCATTAACACAAGAGCAGCT | |
| | TG 3′ | |
| S11 | 5′ GTTTGACAGCTTATCATCGAATAGCTTT | SEQ ID NO: 19 |
| | AATGCGCTAGCTAGACAAGTACTC 3′ | |
| S12 | 5′ GAGTACTTGTCTAGCTAGCGCATTAAAG | SEQ ID NO: 20 |
| | CTATTCGATGATAAGCTGTCAAAC 3′ | |
| S20 | 5′ atgGGCATTTTCTCGAGTCAGCCGGGCC | SEQ ID NO: 14 |
| | ATACCCCGCATCCGGCGGCCagcacaaaaaa | |
| | gaaaccattaac 3′ | |

Repressor variants have been designed in which the selected 12-amino-acid TBD is translationally fused with peptides from cI or cIbZIP. The engineered repressor molecule sequences were initially cloned into the EcoRV site of pETBlue1 (Novagen) such that the ATG start codon was at the optimal distance from the strong ribosome binding site (RBS) in pETBlue1. Digestion with NheI (upstream of the RBS in pETBlue1) and SmaI (downstream of the translational stop) allows mobilization of the engineered coding sequences into vectors containing promoters of different characteristics. Design details of one such vector constructed to contain a weak constitutive promoter are described below.

The crystal structure of the DBD of cI suggested that an insertion at its amino-terminal end, preceding the “arm,” was least likely to interfere with DNA binding. Mutational analysis had clearly indicated that insertions within the helix turn helix would be deleterious to function. At this proposed insertion site (shown in FIG. 2) lysines 3-6 would likely continue to make contact with the backbone of DNA although the affinity of the protein for the DNA might be slightly reduced. It was contemplated that ligand binding would further destabilize the interaction of the engineered repressor with DNA. Deletion of lysines 3-6 is known to disrupt binding to DNA (Eliason et al. PNAS: 82, 2339-2343) and a construct containing this deletion would serve as a negative control.

Oligonucleotides S2 (SEQ ID NO: 13) and S20 (SEQ ID NO: 14) were used to amplify the coding sequence of a temperature sensitive form of cI from lambda cI857ts ind1 DNA (New England Biolabs). Oligonucleotide S2 contained an AvaI site after the translational stop of cI coding sequence to enable blunt-sticky cloning of the PCR product into EcoRV and AvaI digested pETBlue1. Oligonucleotide S20 consists of an ATG start codon followed by a sequence encoding the 12-amino-acid TBD and nucleotides 4-23 of cI. The coding sequence of this engineered repressor is referred to as TBD-cI (SEQ ID NO: 15).

Oligonucleotides S2 (SEQ ID NO: 13) and S8 (SEQ ID NO: 16) were used to amplify the coding sequence of a temperature sensitive form of cI from lambda cI857ts ind1 DNA (New England Biolabs) such that amino acids 2-7 of cI would be deleted. Oligonucleotide S2 contained an AvaI site after the translational stop of cI coding sequence to enable blunt-sticky cloning of the PCR product into EcoRV and AvaI digested pETBlue1. Oligonucleotide S20 consists of an ATG start codon followed by a sequence encoding the 12-amino-acid TBD and nucleotides 22-40 of cI. The coding sequence of this engineered repressor is referred to as TBD-ΔK-cI.

Oligonucleotides S5 and S6 were used to amplify the leucine zipper motif from S. cerevisiae GCN4. Oligonucleotide S5 contained an additional isoleucine at the 5′ end such that ligation of the PCR-product into EcoRV cut pETBlue1 would regenerate the EcoRV site. Digestion of this plasmid by EcoRV followed by ligation to a blunt end PCR product corresponding to amino acids 1-132 of cI (generated by S20 and S7) generated the chimeric repressor TBD-cI-bZIP (SEQ ID NO: 17).

Similarly, ligation of EcoRV cut plasmid to a blunt end PCR product generated by S8 and S7 yielded a chimeric repressor TBD-AK-cI-bZIP (SEQ ID NO: 18), oligonucleotide S8 introducing a deletion of amino acids 2-7 of the cI DBD.

Vectors

Oligonucleotides S11 (SEQ ID NO: 19) and S12 (SEQ ID NO: 20) are complementary to each other and contain the weak constitutive tetracycline resistance promoter. An NheI site has been placed downstream of the promoter sequence followed by ScaI. S11 and S 12 were annealed and ligated into pUniBlunt (Invitrogen) to generate pUnitetpro. Since ScaI is a blunt end cutter, it is compatible with a SmaI (also a blunt end cutter) site downstream of the translational stops for all the repressor constructs in pETBlue1. NheI is present upstream of the RBS (and also repressor coding sequences when present) in pETBlue1. Thus repressor variants can be mobilized from pETBlue1 into pUnitetpro as NheI-SmaI fragments and placed under the control of a tetracycline promoter. This was done for all repressor variants built. It is possible to use a similar strategy to control coding sequences by different promoters such as bla, lac etc.

Moreover, the presence of the loxP sites in pUni enable mobilization of the repressor as well as the promoter controlling it into other vectors with loxH sites through Cre mediated recombination.

The engineered repressors were built as described above and cloned into pUnitetpro. Cre mediated recombination was done with pUnitetpro containing represssors to transfer the repressors (under control of the tet promoter) into pCRT7E (Invitrogen) which has a colE1 origin of replication and can be maintained in the LE392 host strain for subsequent lambda phage infection.

Testing of the Taxol-Responsive Transcriptional Switch

The crystal structure of the DBD of cI suggested that insertions at the N-terminal end might reduce the affinity of its binding to DNA but still allow some level of repressor function. It was contemplated that ligand binding might further destabilize the interaction of the engineered repressor with DNA. The cI-bZIP, TBD-cI-bZIP, and TBD-AK-cI-bZIP constructs were used to test this hypothesis. As described above, cI-bZlPcontains a DBD domain from cI and a bZIP domain from S. cerevisiae GCN4, which was predicted to be functional but not responsive to taxol. TBD-cI-bZIP contains a TBD insertion at the N-terminal end of the construct, which was predicted to reduce repressor function but to be responsive to taxol. TBD-ΔK-cI-bZIP contains a deletion of a lysine rich sequence at the N-terminus of cI known to be involved in interactions with DNA and was predicted to be non-functional.

### Immunity Experiments

There are multiple ways to evaluate lambda repressor function. One such method exploits the central role of the repressor in controlling the decision of lambda phage to enter the lytic or the lysogenic phase. In the presence of a high concentration of functional cI in the bacterial cell, entering lambda phage are pushed into the lysogenic phase. In the absence of cI, bacteria are susceptible to infection by lambda phage: the lysed cells manifest as plaques on a bacterial lawn. The level of functional cI in a cell determines whether incoming phage will continue to enter the lytic cycle or choose the lysogenic cycle, when the phage is integrated into the host genome. Cells with functional cI can thus display immunity to phage superinfection. In addition it is also possible to conduct in vitro experiments, such as electrophoretic mobility shift assays where binding of purified protein to labeled oligonucleotide duplexes can be monitored.

The repressors were placed under the control of a constitutive promoter (tet promoter) in a pUNI (Invitrogen) donor vector. They were transferred to pCRT7 (Invitrogen) for propagation in the bacterial strain LE392 which allows for infection by, and propagation of, phage lambda. Selection was maintained using kanamycin. Strains containing the engineered repressors were infected with lambda phage in the presence and absence of taxol to test for immunity. If the bacterial cells contain functional lambda repressor molecules, then incoming lambda phage cannot establish a lytic cycle and plaque formation is reduced or suppressed. The number and size of the plaques formed on infection with lambda phage is a measure of the immunity.

Five sets of experiments have been done with each of the cI-bZIP, TBD-cI-bZIP, and TBD-ΔK-cI-bZIP constructs. In brief, parallel cultures were grown in the presence of 100 uM taxol or in the absence of taxol. Cells were incubated with standardized dilutions of lambda phage at 30° C. for 30 minutes and plated with top agar on lambda plates with kanamycin. For the cultures grown with 100 uM taxol, the top agar also contains 100 uM taxol. Plaque phenotype was scored at 24 and 48 hours of incubation at 30° C. The number of plaques was counted for three experiments using 3-5 replicates for each set.

Cells containing cI-bZIP on infection with lambda phage gave rise to miniscule plaques barely visible to the eye. The phenotype was not changed by the addition of taxol.

Cells containing TBD-cI-bZIP on infection with lambda phage gave rise to very small plaques. On addition of taxol, the plaque size was increased while the number of plaques was not significantly altered, indicating that taxol indeed modulates the DNA-binding activity of the engineered taxol-responsive transcriptional repressor.

Cells containing TBD-AK-cI-bZIP on infection with lambda phage gave rise to large plaques. There was no alteration in the number and size of the plaques on the addition of taxol.

DNA Binding Experiments

Repressor molecules as described above are modified to contain a His6 tag in the linker region and placed under the control of the strong inducible T7 promoter. The modified repressor variants are purified and tested for direct binding to fluorescently labeled oligo duplexes corresponding to operator binding sites of lambda repressor. The in vitro binding assays are designed with or without taxol and the results are compared to test whether taxol directly affects the DNA binding affinity of lambda repressor.

### INCORPORATION BY REFERENCE

Each document cited hereinabove is expressly incorporated herein by reference.

We claim:

1-66. (canceled)

67. A method of engineering a ligand-responsive chimeric protein construct that modulates gene expression responsive to the presence, concentration, or absence of a preselected ligand, the method comprising the steps of:
identifying one or more amino acid sequences that bind a preselected ligand using a recombinant display technique selected from the group consisting of phase display, retroviral display, bacterial surface display, yeast surface display, ribosome display, two-hybrid techniques, three-hybrid techniques, and derivatives thereof;
designing, based on the one or more amino acid sequences, an engineered peptide that binds the preselected ligand;
selecting an interaction domain which binds to a target biomolecule to modulate expression of a gene;
identifying a permissive position within or adjacent the interaction domain at which insertion of a heterologous peptide permits retention of binding of the interaction domain to the target biomolecule and conserves expression modulation activity; and
synthesizing a construct comprising the engineered peptide fused to the interaction domain at the permissive position,
thereby to produce a construct wherein binding of the ligand to the engineered peptide causes a change in said chimeric protein, the change regulating binding of the interaction domain to the target biomolecule and expression.

68. (canceled)

69. A method as in claim 67, wherein the engineered peptide is among the one or more amino acid sequences identified using the recombinant display technique.

70. A method as in claim 67, wherein the engineered peptide reflects a consensus sequence derived from the one or more amino acid sequences identified.

71. A method as in claim 67, wherein the engineered peptide is no more than one hundred amino acids in length.

72. A method of engineering a stimulus-responsive chimeric protein construct which modulates expression of a preselected gene, the method comprising the steps of:
identifying a stimulus-responsive peptide of no more than one hundred amino acids in length;
selecting an interaction domain capable of binding to a target biomolecule to modulate transcription of a preselected gene;
identifying a permissive position within or adjacent the interaction domain at which insertion of a heterologous peptide permits binding of the interaction domain to the target biomolecule; and
synthesizing a construct comprising the stimulus-responsive peptide fused to the interaction domain at the permissive position,
thereby to produce a construct wherein recognition of the stimulus causes change in said chimeric protein, the change regulating binding of the interaction domain to the target biomolecule.

73. A method as in claim 71 or 72, wherein the engineered peptide is no more than eighty amino acids in length.

74. A method as in claim 71 or 72, wherein the engineered peptide is no more than sixty amino acids in length.

75. A method as in claim 71 or 72, wherein the engineered peptide is no more than forty amino acids in length.

76. A method as in claim 71 or 72, wherein the engineered peptide is no more than twenty amino acids in length.

77. A method as in claim 67 or 72, wherein the permissive position is identified using stereochemical data about the three-dimensional structure of the interaction domain.

78. A method as in claim 67 or 72, wherein the permissive position is identified using mutational data about the interaction domain.

79. A method of engineering a stimulus-responsive chimeric protein construct, the method comprising the steps of:
identifying, from a database of information, a stimulus-responsive protein;
selecting an interaction domain capable of binding to a target biomolecule;
identifying a permissive position within or adjacent the interaction domain at which insertion of a heterologous peptide permits retention of binding of the interaction domain to the target biomolecule; and
synthesizing a construct comprising the stimulus-responsive protein, or a peptide derivative thereof, fused to the interaction domain at the permissive position,
thereby to produce a construct wherein receipt of the stimulus by the stimulus-responsive protein, or a peptide derivative thereof causes change in said chimeric protein, the change regulating binding of the interaction domain to the target biomolecule.

80-97. (canceled)

98. The construct produced by the method of claim 67.

99. The construct produced by the method of claim 72.

100. The construct produced by the method of claim 79.


SEQUENCE LISTING (20 sequences)

SEQ ID NO: 1 - protein, 21 aa, Artificial
  Zinc finger consensus sequence
  misc_feature (2)..(3): wherein Xaa at positions 2, 3 can be any amino acid
  misc_feature (5)..(7): wherein Xaa at positions 5, 6, 7 can be any amino acid
  misc_feature (9)..(13): wherein Xaa at positions 9, 10, 11, 12, 13 can be any amino acid
  misc_feature (15)..(16): wherein Xaa at positions 15, 16 can be any amino acid
  misc_feature (18)..(20): wherein Xaa at positions 18, 19, 20 can be any amino acid
CXXCXXXFXXXXXLXXHXXXH

SEQ ID NO: 2 - protein, 22 aa, Artificial
  Zinc finger consensus sequence
  misc_feature (2)..(4): wherein Xaa at positions 2, 3, 4 can be any amino acid
  misc_feature (6)..(8): wherein Xaa at positions 6, 7, 8 can be any amino acid
  misc_feature (10)..(14): wherein Xaa at positions 10, 11, 12, 13, 14 can be any amino acid
  misc_feature (16)..(17): wherein Xaa at positions 16, 17 can be any amino acid
  misc_feature (19)..(21): wherein Xaa at positions 19, 20, 21 can be any amino acid
CXXXCXXXFXXXXXLXXHXXXH

SEQ ID NO: 3 - protein, 23 aa, Artificial
  Zinc finger consensus sequence
  misc_feature (2)..(5): wherein Xaa at positions 2, 3, 4, 5 can be any amino acid
  misc_feature (7)..(9): wherein Xaa at positions 7, 8, 9 can be any amino acid
  misc_feature (11)..(15): wherein Xaa at positions 11, 12, 13, 14, 15 can be any amino acid
  misc_feature (17)..(18): wherein Xaa at positions 17, 18 can be any amino acid
  misc_feature (20)..(22): wherein Xaa at positions 20, 21, 22 can be any amino acid
CXXXXCXXXFXXXXXLXXHXXXH

SEQ ID NO: 4 - protein, 21 aa, Artificial
  Zinc finger consensus sequence
  misc_feature (2)..(3): wherein Xaa at positions 2, 3 can be any amino acid
  misc_feature (4)..(16): wherein Xaa at positions 5, 6, 7, 8, 9, 10, 11, 12, 13, 14, 15, 16, 17 can be any amino acid
  misc_feature: wherein Xaa at positions 19, 20 can be any amino acid
CXXCXXXXXXXXXXXXXCXXC

SEQ ID NO: 5 - protein, 7 aa, Artificial
  target sequence for protein kinase A
LRRASLG

SEQ ID NO: 6 - protein, 10 aa, Artificial
  substrate for casein kinase II
RRREEETEEE

SEQ ID NO: 7 - protein, 12 aa, Artificial
  substrate sequence for v-Abl tyrosine kinase
EAIYAAPFAKKK

SEQ ID NO: 8 - DNA, 27 bp, Artificial
  primer for leucine zipper motif
atcgcgcacatgaaacaacttgaagac

SEQ ID NO: 9 - DNA, 22 bp, Artificial
  primer for leucine zipper motif
tcagcgttcgccaactaatttc

SEQ ID NO: 10 - DNA, 26 bp, Artificial
  primer for lambda repressor
atgagcacaaaaaagaaaccattaac

SEQ ID NO: 11 - DNA, 18 bp, Artificial
  primer for lambda repressor
gcttacccagcgctccgc

SEQ ID NO: 12 - DNA, 504 bp, Artificial
  cI-bZIP repressor variant
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagcgctgggtaagcatcgcgcacatgaaacaacttgaagacaaggttgaagaattgctttcgaaaaattatcacttggaaaatgaggttgccagattaaagaaattagttggcgaacgctga

SEQ ID NO: 13 - DNA, 35 bp, Artificial
  primer for coding sequence of a temperature sensitive form of thelambda repressor containing an AvaI sit
ttacaacgcccgggtcagccaaacgtctcttcagg

SEQ ID NO: 14 - DNA, 71 bp, Artificial
  primer for the coding sequence of a temperature sensitive form oflambda represso
atgggcattttctcgagtcagccgggccataccccgcatccggcggccagcacaaaaaagaaaccattaac

SEQ ID NO: 15 - DNA, 784 bp, Artificial
  TBD-cI chimeric repressor variant
atgggcattttctcgagtcagccgggccataccccgcatccattaacacaagagcagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttcatcaccatcaccatcacttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 16 - DNA, 61 bp, Artificial
  primer for coding sequence of a temperature sensitive form of lambda repressor
atgggcattttctcgagtcagccgggccataccccgcatccattaacacaagagcagcttg

SEQ ID NO: 17 - DNA, 542 bp, Artificial
  TBD-cI-bZIP chimeric repressor variant
atgggcattttctcgagtcagccgggccataccccgcatccattaacacaagagcagcacaaaaaagaaaccattaacaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagcgctgggtaagcatcgcgcacatgaaacaacttgaagacaaggttgaagaattgctttcgaaaaattatcacttggaaaatgaggttgccagattaaagaaattagttggcgaacgctga

SEQ ID NO: 18 - DNA, 525 bp, Artificial
  TBP-cI-bZIP repressor variant
atgggcattttctcgagtcagccgggccataccccgcatccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagcgctgggtaagcatcgcgcacatgaaacaacttgaagacaaggttgaagaattgctttcgaaaaattatcacttggaaaatgaggttgccagattaaagaaattagttggcgaacgctga

SEQ ID NO: 19 - DNA, 52 bp, Artificial
  primer containing sequence for a weak tetracycline resistance promote
gtttgacagcttatcatcgaatagctttaatgcgctagctagacaagtactc

SEQ ID NO: 20 - DNA, 52 bp, Artificial
  primer containing sequence for a weak constitutive tetracycline promote
gagtacttgtctagctagcgcattaaagctattcgatgataagctgtcaaac